(19) United States
(12) Patent Application Publication
(10) Pub. No.: US 2024/0352544 A1
(43) Pub. Date: Oct. 24, 2024

(54) COMPOSITIONS, KITS, AND METHODS FOR DETECTION OF NUCLEIC ACID SEQUENCE LOADS

(71) Applicant: Life Technologies Corporation, Carlsbad, CA (US)

(72) Inventors: Say Li Kong, Singapore (SG); Manohar Furtado, San Ramon, CA (US); Daryn Kenny, Pleasant Hill, CA (US); Yu Hui Grace Wong, Singapore (SG); San Min Leow, Singapore (SG); Andy Ying, Fremont, CA (US)

(21) Appl. No.: 18/294,322

(22) PCT Filed: Aug. 2, 2022
(86) PCT No.: PCT/US2022/039194

§ 371 Date: Feb. 1, 2024

Related U.S. Application Data
(60) Provisional application No. 63/228,527, filed on Aug. 2, 2021.

(51) Int. Cl. C12Q 1/70; C12Q 1/6851

(52) U.S. Cl. CPC C12Q 1/701; C12Q 1/6851

(57) ABSTRACT

Disclosed are compositions, kits, and methods for quantifying a target nucleic acid from a sample. Compositions, kits, and methods enable the comparison of target nucleic acid loads between two or more test samples by normalizing measured levels (using a standard curve) of the target nucleic acid in each sample according to relative levels of endogenous nucleic acid in each test sample.

### PRIORITY

This PCT application claims priority to and the benefit of U.S. Provisional Patent Application Ser. No. 63/228,527, filed on Aug. 2, 2021. The contents of the foregoing application, in their entirety, are incorporated herein by reference.

### FIELD

The present teachings relate to compositions, kits, and methods for quantifying a target nucleic acid from a sample. In particular, embodiments described herein enable the comparison of target nucleic acid “loads” between two or more test samples by normalizing measured levels of the target nucleic acid in each sample according to relative levels of endogenous nucleic acid in each test sample.

### BACKGROUND

Assays to detect target nucleic acid sequences of interest are widely used in molecular biology and medicine. Clinical applications typically involve collection of sample from a subject, extraction of nucleic acid, and subjecting the extracted sample to amplification conditions in the presence of target-specific primers. The presence of amplification products thus indicates that the target nucleic acid was present within the sample, whereas failure to measure amplification products indicates that the target nucleic acid was absent or was present in levels too low for detection. Such assays are therefore useful for detecting and monitoring pathogenic diseases.

In some applications, samples used in such assays are sourced from the subject's blood. Blood samples, however, are relatively difficult to obtain. In applications of pathogenic disease monitoring, for example, subjects are less likely to comply with a recommendation to be tested or volunteer to be tested if sample collection involves drawing blood. In addition, while there may be a positive association between viral load, as measured in blood serum, and disease severity, the presence of pathogenic nucleic acid (e.g., viral RNA) in blood seems to appear only in a minority of patients, and when it is present, there is often significant heterogeneity across studies. Therefore, in some cases, there is no conclusive evidence of this association if the samples are soured from blood. Many assays are therefore designed to utilize saliva samples or samples collected via swabs (e.g., oropharyngeal or nasopharyngeal).

While saliva and/or swab-based samples are easier to obtain, there are drawbacks associated with their use. In particular, the amount and concentration of organic matter in a saliva or swab-based sample can vary widely from sample to sample, even from the same individual. Differences are due to different sample collection techniques, different overall mass or volume collected from sample to sample, differences in subject physiology or anatomy, and differences in sample collection equipment, for example.

Inconsistencies in levels of organic matter from sample to sample make it difficult to generate meaningful comparisons of the total load of target nucleic acid between samples, even for samples from a single subject. In many applications it would be useful to determine how the load of target nucleic acid in a subject is changing over time, such as for monitoring the progression of a pathogenic disease. In the current state of the art, however, meaningful comparisons of this type cannot be made due to excessive variation between samples. For example, where testing of two different samples results in measured absolute quantities of target nucleic acid, it cannot be concluded that one sample actually demonstrates lower or higher levels of the target nucleic acid compared to the other, as any differences (or even coincidental similarities) may actually be caused by differences in the amount or concentration of organic matter collected in the samples.

Accordingly, there is an ongoing need for systems, methods, kits, and other embodiments that quantify a target nucleic acid from a sample and enable comparison of target nucleic acid loads between two or more test samples.

### SUMMARY

An embodiment of the invention includes a method for quantifying a target nucleic acid across multiple samples, the method comprising providing two or more test samples each including the target nucleic acid; providing a set of control samples each having a known concentration of a control nucleic acid; amplifying at least a portion of the target nucleic acid in each of the test samples by subjecting each of the test samples to amplification conditions in the presence of target-specific primers; amplifying at least a portion of the control nucleic acid in each of the control samples by subjecting each of the control samples to amplification conditions in the presence of control primers; generating a standard curve using the results of amplifying the control nucleic acid; determining an absolute quantity (AQ) of the target nucleic acid in each of the test samples using the standard curve; amplifying an endogenous control nucleic acid in each of the test samples by subjecting each of the test samples to amplification conditions in the presence of endogenous sequence primers; determining a correction factor (RQ) for each of the test samples based on relative levels of endogenous nucleic acid in a respective one of the test samples; and determining a corrected quantity (corrected AQ) of the target nucleic acid in each of the test samples by normalizing the absolute quantity of the target nucleic acid using the correction factor of the respective one of the test samples.

In an example embodiment, the two or more test samples are each derived from the same subject.

In an example embodiment, the two or more test samples are obtained at different times and/or from different locations of the subject.

In an example embodiment, at least two of the different times are separated by a time period of 1 hour, 2 hours, 3 hours, 6 hours, 12 hours, 24 hours, 48 hours, 72 hours, 96 hours, one week, two weeks, three weeks, four weeks, six weeks, eight weeks, ten weeks, three months, four months, five months, six months, or a time period range with endpoints defined by any two of the foregoing values.

In an example embodiment, the target nucleic acid is a viral nucleic acid. In another example embodiment, the target nucleic acid is a SARS-CoV-2 nucleic acid.

In an example embodiment, the test samples can be derived from swab samples. In an example embodiment, the swab samples are nasal swab samples. In another example embodiment, each of the nasal swab samples are from a same nostril of the subject.

In an example embodiment, the method further comprises extracting the target nucleic acid from the swab samples prior to subjecting each of the test samples to amplification conditions.

In an example embodiment, the control nucleic acid in each of the control samples comprise a nucleic acid comprising the target nucleic acid.

In an example embodiment, the control nucleic acid in each of the control samples comprise a whole or partial viral genome.

In an example embodiment, the control nucleic acid in each of the control samples is capable of being amplified with the target-specific primers.

In an example embodiment, the control primers and the target-specific primers are the same.

In an example embodiment, the endogenous sequence primers are specific for RNase P.

In an example embodiment, the target-specific primers are specific to one or more of the Orf1a gene, the Orf1b gene, the N gene, or the S gene of SARS-CoV-2.

In an example embodiment, the test samples exclude blood samples.

In an example embodiment, the test samples are nasopharyngeal or oropharyngeal samples.

In an example embodiment, the method further comprises establishing a baseline correction factor for a first test sample associated with a first time point, and determining subsequent correction factors for subsequent test samples relative to the baseline correction factor.

In an example embodiment, the method further comprises determining a corrected quantity of the subsequent test samples relative to the first test sample to illustrate relative change in target nucleic acid load of the test samples over time.

In an example embodiment, the amplifying of the at least a portion of the target nucleic acid in each of the test samples comprises a reverse transcription reaction.

In an example embodiment, the amplifying of the at least a portion of the control nucleic acid in each of the control samples comprises a reverse transcription reaction.

In an example embodiment, the amplifying of the endogenous nucleic acid in each of the test samples exclude a reverse transcription reaction.

In an example embodiment, the amplification of the control nucleic acid in each of the control samples and amplification of the endogenous control nucleic acid in each of the test samples have substantially similar efficiency.

In an example embodiment, amplification of the control nucleic acid in each of the control samples and amplification of the endogenous control nucleic acid in each of the test samples have efficiencies that differ by no more than about 6%, no more than about 5%, or no more than about 4%.

In an example embodiment, an efficiency plot of amplification of the control nucleic acid in each of the control samples and an efficiency plot of amplification of the endogenous control nucleic acid in each of the test samples have slopes (Cq/quantity) that differ by no more than about 6%, no more than about 5%, or no more than about 4%.

Another embodiment of the present invention includes a method for quantifying a target viral nucleic acid from a sample, the method comprising providing two or more test samples each including the target viral nucleic acid; providing a set of control samples each having a known concentration of the target viral nucleic acid; amplifying at least a portion of the target viral nucleic acid in each of the test samples by subjecting each of the test samples to amplification conditions in the presence of target-specific primers; amplifying at least a portion of the target viral nucleic acid in each of the control samples by subjecting each of the control samples to amplification conditions in the presence of the target-specific primers; generating a standard curve using the results of amplifying the target viral nucleic acid in each of the control samples; determining an absolute quantity of the target viral nucleic acid in each of the test samples using the standard curve; amplifying an endogenous nucleic acid in each of the test samples by subjecting each of the test samples to amplification conditions in the presence of endogenous sequence primers; determining a correction factor for each of the test samples based on relative levels of endogenous nucleic acid in a respective one of the test samples; and determining a corrected quantity of the target viral nucleic acid in each of the test samples by normalizing the absolute quantity of the target viral nucleic acid using the correction factor for the respective one of the test samples.

In an example embodiment, the two or more test samples are each derived from the same subject.

In an example embodiment, the two or more test samples are obtained at different times.

In an example embodiment, at least two of the different times are separated by a time period of 1 hour, 2 hours, 3 hours, 6 hours, 12 hours, 24 hours, 48 hours, 72 hours, 96 hours, one week, two weeks, three weeks, four weeks, six weeks, eight weeks, ten weeks, three months, four months, five months, six months, or a time period range with endpoints defined by any two of the foregoing values.

In an example embodiment, the target viral nucleic acid is a SARS-CoV-2 nucleic acid.

In an example embodiment, the test samples are derived from swab samples.

In an example embodiment, the swab samples are nasal swab samples.

In an example embodiment, each of the nasal swab samples is from a same nostril of the subject.

In an example embodiment, the method further comprises extracting the target nucleic acid from the swab samples prior to subjecting each of the test samples to the amplification conditions.

In an example embodiment, the endogenous sequence primers are specific for RNase P.

In an example embodiment, the target-specific primers are specific to one or more of the Orf1a gene, the Orf1b gene, the N gene, or the S gene of SARS-CoV-2.

In an example embodiment, the test samples exclude blood samples.

In an example embodiment, the test samples are nasopharyngeal or oropharyngeal samples.

In an example embodiment, the method further comprises establishing a baseline correction factor for a first test sample associated with a first time point; and determining subsequent correction factors for subsequent and/or additional test samples relative to the baseline correction factor.

In an example embodiment, the method further comprises determining a corrected quantity of the subsequent test samples relative to the first test sample to illustrate relative change in target nucleic acid load of the test samples over time.

In an example embodiment, the amplifying of at least a portion of the target nucleic acid in each of the test samples comprises a reverse transcription reaction.

In an example embodiment, the amplifying of at least a portion of the target nucleic acid in each of the control samples comprises a reverse transcription reaction.

In an example embodiment, the amplifying of the endogenous nucleic acid in each of the test samples excludes performing a reverse transcription reaction.

In an example embodiment, the amplifying of the target nucleic acid in each of the control samples and the amplifying of the endogenous nucleic acid in each of the test samples have substantially similar efficiency.

In an example embodiment, an efficiency plot of amplification of the target nucleic acids in each of the control samples and an efficiency plot of amplification of the endogenous nucleic acid in each of the test samples have slopes (Cq/quantity) that differ by no more than about 6%, no more than about 5%, or no more than about 4%.

### BRIEF DESCRIPTION OF THE DRAWINGS

FIG. 1A is a schematic illustration of the SARS-CoV-2 virion structure.

FIG. 1B is a schematic diagram of the RNA genome of SARS-CoV-2, illustrating potential target regions that assays described herein may be targeted toward.

FIG. 2 illustrates a method for quantifying a target nucleic acid from a sample.

FIG. 3 is an amplification efficiency plot illustrating similar PCR efficiency between the N/S SARS-CoV-2 gene targets and RNase P target using a TaqCheck™ SARS-CoV-2 Fast PCR Assay Kit.

FIGS. 4A and 4B are amplification plots of various control sample dilutions of the N/S gene and RNase P, respectively, using a TaqCheck™ SARS-CoV-2 Fast PCR Assay Kit.

FIGS. 5A through 5D illustrate amplification plots of various control sample dilutions of the N gene, Orf1a gene, Orf1b gene targets and RNase P target, respectively, using a TaqMan™ SARS-CoV-2 with RNase P Assay 2.0.

FIGS. 5E and 5F are variability charts of mean Cq values for the different control sample dilutions for the Orf1a gene and the RNase P target, respectively, showing potential crosstalk between dye channels at high copy number samples.

FIG. 6 illustrates the standard curve utilized to obtain “COVID AQ” values from the Cq values resulting from PCR.

FIG. 7 illustrates the change in the Log (Quant.) over time from the initial T₀ time period

### DETAILED DESCRIPTION

All publications and patent applications cited herein, as well as the Appendices attached hereto, are incorporated by reference in their entirety for all purposes to the same extent as if each individual Appendix, publication or patent application were specifically and individually indicated to be so incorporated by reference. Although the Appendices attached hereto may include particular examples that reference specific target nucleic acids, formulations, and process steps, it will be understood that these examples may be modified by using any of the formulations, components, and/or process steps described elsewhere herein.

When an example “embodiment” or a particular “assay” is described herein, it will be understood that the features of the embodiment may be applicable to a composition (e.g., the particular physical components of an assay such as primers and/or probes), a kit (e.g., primers and/or probes and additional buffers, reagents, etc.), or a method (e.g., a process for detecting and/or quantifying target nucleic acids) as appropriate. For simplicity, many embodiments are presented by describing “assays”, but it will be understood that the associated methods of using the assays are also intended to form part of this disclosure.

### Overview of Compositions, Kits, and Methods for Quantifying a Target Nucleic Acid

As discussed above, there are challenges associated with comparing measured quantity of a target nucleic acid between different samples, even when the samples are sourced from the same subject. These challenges are particularly acute in applications that utilize non-blood clinical samples, such as in applications where oropharyngeal and/or nasopharyngeal swabs are utilized during sample collection. Challenges include difficulties in monitoring the change in target nucleic acid load in the subject over time, for example. Although information regarding change in loads over time would be useful in many applications (e.g., in diagnosing and/or monitoring progression of a pathogenic disease), the concomitant difficulties have thus far prevented the ability to readily obtain and utilize such information. It is difficult to make meaningful comparisons across different samples because changes in actual levels of the target nucleic acid may be obfuscated by inter-sample differences in amount or concentration of collected organic matter (e.g., the subject's cells). Any actual changes in levels of the target nucleic acid in the subject are thus confounded by differences between samples.

Described herein are compositions, kits, and methods configured to quantify target nucleic acids and to enable meaningful comparisons between multiple test samples by normalizing measured levels of the target nucleic acid in each sample according to relative levels of endogenous nucleic acid in each test sample. Embodiments described herein beneficially enable improved diagnosis and/or monitoring of disease progression in a subject over time, allowing medical professionals to better determine whether target nucleic acid associated with the pathogen is increasing, decreasing, or staying the same within the subject over time. This information can improve disease diagnosis, treatment, and/or prognosis by better illustrating treatment effects, highlighting risk thresholds, and/or indicating outcome probabilities, for example.

### Target Nucleic Acids

As disclosed herein, the target nucleic acid may be viral, bacterial, fungal, or eukaryotic. The target nucleic acid may be from a pathogen. In embodiments related to pathogenic disease diagnosis and/or monitoring, the target pathogen can be any pathogen that leaves detectable levels of nucleic acid within the subject because of infection of the subject. The pathogen may be a virus, bacteria, fungus, or eukaryotic parasite. Embodiments described herein are particularly useful where target nucleic acid associated with the pathogen is obtainable through saliva collection and/or through a swab-based collection process. Swab-based collection processes commonly involve nasopharyngeal or oropharyngeal swabs, though certain embodiments are applicable to other types of swab samples, such as cheek swabs, wound swabs, skin swabs, aural swabs, anal swabs, vaginal swabs, or swabs of other anatomical locations.

Respiratory pathogens in particular may be diagnosed and/or monitored using swab-based sample collection processes (usually nasopharyngeal or oropharyngeal swabs). Examples of respiratory microorganisms that may be targeted are listed in Table 1, below. Assays targeting one or more of the below organisms (and/or other organisms) include appropriate primers to enable amplification of target nucleic acid sequences associated with the pathogens, and optionally one or more probes to aid in detection of amplification products that target one or more newly emerging pathogens or microorganisms of interest.

TABLE 1
Example Respiratory Microorganisms

| | | | | |
|---|---|---|---|---|
| AdV 1 | FluB | CoV HKU1 | Parecho V | M. catarrhalis |
| | (pan) | | | |
| HBOV | hPIV1 | CoV NL63 | Bordetella | M. pneumoniae |
| | | | spp. | |
| HHV3 | hPIV2 | CoV OC43 | B. holmesii | S. aureus |
| HHV4 | hPIV3 | Mumps | B. pertussis | S. pneumoniae |
| HHV5 | hPIV4 | MERS-COV | C. pneumoniae | P. jirovecii |
| HHV6 | RSVA | SARS-COV | H. influenzae | |
| FluA | RSVB | SARS-COV-2 | K. pneumoniae | |
| (pan) | | | | |
| FluA (H1) | hMPV | Entero V (pan) | L. pneumophila | |
| FluA (H3) | Measles | Entero V D68 | | |
| | CoV 229E | Rhino V 1of2 | | |
| | | Rhino V 2of2 | | |

For a selected pathogen or other target organism, one or more nucleic acids may be targeted by designing target-specific primers that enable amplification of the target nucleic acid when the sample and the target-specific primers are subjected to amplification conditions. One of skill in the art is equipped to design appropriate primers. Such methods are described in Basu, Chhandak (Ed.) “PCR Primer Design” (Methods in Molecular Biology) 2^{nd} edition (2015).

Although not limited to any particular pathogen or set of pathogens, certain embodiments are configured for quantifying target nucleic acids associated with coronaviruses, in particular the SARS-CoV-2. The SARS-CoV-2 virus, also known as 2019-nCoV, is associated with the human respiratory disease COVID-19. The virus isolated from early cases of COVID-19 was provisionally named 2019-nCoV. The Coronavirus Study Group of the International Committee on Taxonomy of Viruses has subsequently given the official designation of SARS-CoV-2. For the purposes of this disclosure SARS-CoV-2 and 2019-nCoV are considered to refer to the same virus.

The genetic sequence of the initially characterized “reference” form of SARS-CoV-2 is based on the sequence associated with NCBI accession no. NC_045512.2 (see GenBank: MN908947.3) which describes a genome of 29,903 base pairs. Because SARS-CoV-2 is an RNA virus, it can mutate with relatively high frequency, which has led to the emergence of new variants, and it is likely that additional variants will continue to emerge over time.

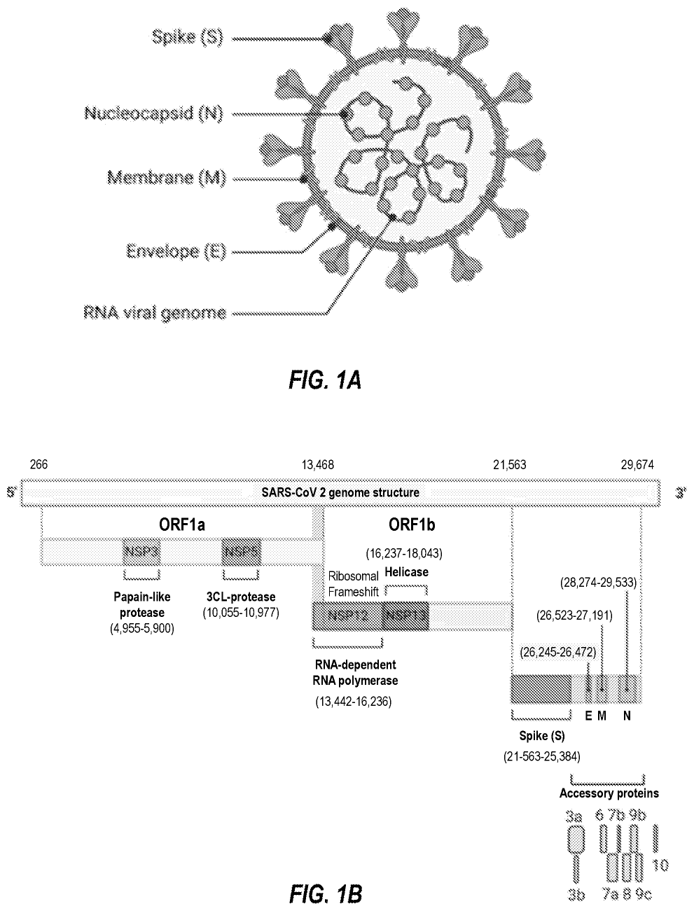

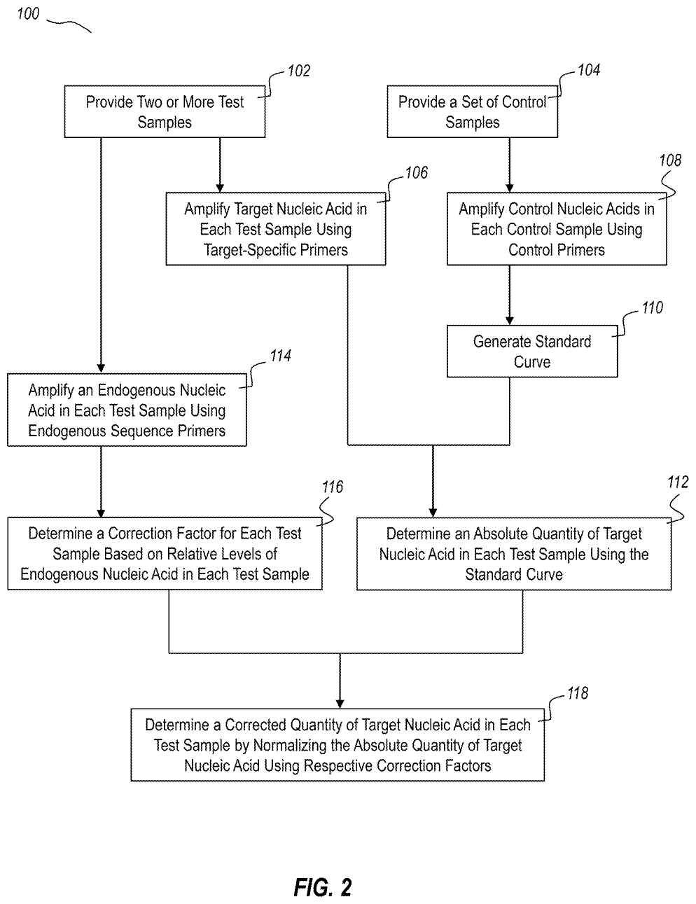

FIG. 1A is a schematic of the SARS-CoV-2 virion, and FIG. 1B is a diagram of the SARS-CoV-2 RNA genome showing particular regions that may be targeted by selecting appropriate, target-specific primers. As shown, potential target genes include the Orf1a, Orf1b, S, E, M, and N genes, among several other accessory proteins. The SARS-CoV-2 genome encodes two large genes Orf1a and Orf1b, which encode 16 non-structural proteins (NSP1-NSP16). These NSPs are processed to form a replication-transcription complex (RTC) that is involved in genome transcription and replication. The structural genes encode the structural proteins, spike(S), envelope (E), membrane (M), and nucleocapsid (N). The accessory proteins are unique to SARS-CoV-2 in terms of number, genomic organization, sequence, and function.

Table 2 illustrates some of the mutations that have occurred in the SARS-CoV-2 genome, as well as their associated effects and epidemiological impacts, where known. The numbering system used to designate these mutations is based on the “reference” sequence as defined above. For example, the mutation “S.N501Y.A_T” refers to a mutant form of the spike(S) protein wherein amino acid residue no. 501 is changed from asparagine (N) to tyrosine (Y). The latter portion of the label “A_T” illustrates that the mutation is also associated with a change from an adenine (A) to a thymine (T). Note that RNA comprises uracil (U), but notation included herein may sometimes simply refer to the corresponding DNA base pair thymine (T). The initial part of the label specific to the protein involved and/or the latter portion of the label specific to the nucleotide mutation may occasionally be dropped from the label for convenience. Those with skill in the art will readily recognize the mutation nomenclature used herein.

TABLE 2
SARS-COV-2 Mutations

| Mutation Description | Gene | Effects on Virus | Epidemiological Impact |
|---|---|---|---|
| S.delH69V70 | S | Conformation Spike | 1) evade immune response, |
| | | Protein; N terminal | 2) assays targeting S gene |
| | | domain of spike | may not pick up |
| S.N501Y.A_T | S | Located within receptor | 1) fast growing lineage, 2) |
| | | binding domain one of 6 | increased binding affinity to |
| | | key contact residues | hACE2 receptor in cells |
| S.E484K.G_A | S | Mutation in RBD region | reduces antibody |
| | | | recognition; interface with |
| | | | ACE2 receptor |
| S.K417N.G_C | S | Mutation to RBD region | mutation in RBD region not |
| | | (in South African | present in UK strain, may |
| | | variant) | impact vaccine effectiveness |
| S.N439K.C_A | S | Impacts ability to evade | 1) increased binding affinity |
| | | antibody-mediated | to hACE2 receptor, 2) evade |
| | | immunity | some Mabs |
| S.D614G.A_G | S | Ability to spread more | Transmissibility impact |
| | | quickly | moderate |
| S.A222V.C_T | S | 20A.EU1 SARS-COV-2 | 1) fast growing lineage, 2) |
| | | cluster (lineage B.1.177) | no impact from mutation |
| S.Y453F.A_T | S | Associated with minks | 1) increased binding affinity |
| | | | to hACE2 receptor, 2) evade |
| | | | some Mabs |
| S.P681H.C_A | S | Near highly variable | Near furin S1/S2 cleavage |
| | | S1/S2 furin cleavage site | site. Believed to be the |
| | | | dominant global variant. |
| orf8.Q27stop | Orf* | Q27 stop codon | Truncated Orf8 gene; allows |
| | | | downstream mutations to |
| | | | accrue |
| S.delY144 | S | Conformation Spike | 1) evade immune response, |
| | | Protein; N terminal | 2) assays targeting S gene |
| | | domain of spike | may not pick up |
| S.A570D.C_A | S | Located within receptor | 1) fast growing lineage, 2) |
| | | binding domain; one of | Increased binding affinity to |
| | | 6 key contact residues, | hACE2 receptor in cells |
| S.T716I.C_T | S | Mutation in RBD region | Reduces antibody |
| | | | recognition; interface with |
| | | | ACE2 receptor |
| S.S982A.T_G | S | Mutation to RBD region | Mutation in RBD region not |
| | | in South African variant | present in UK strain, may |
| | | | impact vaccine effectiveness |
| S.D1118H.G_C | S | Impacts ability to evade | 1) increased binding affinity |
| | | antibody-mediated | to hACE2 receptor, 2) evade |
| | | immunity | some Mabs |
| orf1.A1708D.C_A | Orfla | Ability to spread more | Transmissibility impact |
| | | quickly | moderate |
| S.A701V.C_T | S | 20A.EU1 SARS-COV-2 | 1) fast growing lineage, 2) |
| | | cluster (lineage B.1.177) | no impact from mutation |
| S.del22286_9bp | S | | 1) increased binding affinity |
| | | | to hACE2 receptor, 2) evade |
| | | | some Mabs |
| S.D80A.A_C | S | | Near furin S1/S2 cleavage |
| | | | site. Believed to be the |
| | | | dominant global variant. |
| S.D215G.A_G | S | | Truncated Orf8 gene; allows |
| | | | downstream mutations to |
| | | | accrue |
| S.L18F.C_T | S | | 1) evade immune response, |
| | | | 2) assays targeting S gene |
| | | | may not pick up |
| S.R246I.G_T | S | | 1) fast growing lineage, 2) |
| | | | increased binding affinity to |
| | | | hACE2 receptor in cells |

Certain embodiments are directed to quantifying a target SARS-CoV-2 nucleic acid. In some embodiments, one or more target-specific primers are targeted to a SARS-CoV-2 nucleic acid corresponding to reference SARS-CoV-2. In some embodiments, one or more target-specific primers are targeted to a SARS-CoV-2 nucleic acid corresponding to an existing or future variant form of SARS-CoV-2. Embodiments may target one (e.g., in a singleplex reaction) or more (e.g., in a multiplex reaction) nucleic acid sequences associated with any of the SARS-CoV-2 genes described herein. Typically, embodiments target one or more nucleic acid sequences associated with the Orf1a, Orf1b, S, or N gene.

Exemplary assays suitable for targeting SARS-CoV-2 include TaqCheck™ SARS-CoV-2 Fast PCR Assay Kit (Thermo Fisher Scientific, Catalog No. A47693), TaqPath™ COVID-19 Combo Kit (Thermo Fisher Scientific), TaqPath™ COVID-19 Combo Kit Advanced (Thermo Fisher Scientific, Catalog No. A47814), TaqMan™ SARS-CoV-2 with RNase P Assay 2.0 (Thermo Fisher Scientific, Catalog No. A51121), TaqMan™ SARS-CoV-2 RNase P Assay Kit (Thermo Fisher Scientific), CoviPath™ COVID-19 RT PCR Kit (Thermo Fisher Scientific), and TaqMan™ SARS-CoV-2 Fast PCR Combo Kit 2.0 (Thermo Fisher Scientific, Catalog No. A51607).

### Exemplary Assay 1

A first exemplary assay is a multiplex real-time RT-PCR assay for the detection of viral RNA in samples. The multiplex real-time RT-PCR assay can be for the detection of SARS-CoV-2 viral RNA in human raw saliva samples, for example, the TaqCheck™ SARS-CoV-2 Fast PCR Assay Kit (Thermo Fisher Scientific).

The first exemplary assay comprises forward and reverse primers specific to a target viral nucleic acid (e.g., the N/S SARS-CoV-2 gene targets), and an endogenous nucleic acid (e.g., RNase P gene target). The first exemplary assay further comprises a fluorescent or other detectable label (e.g., VIC dye) for detecting the target viral nucleic acid, and another fluorescent or other detectable label (e.g., FAM dye) for detecting the endogenous nucleic acid. The first exemplary assay also comprises a quencher (e.g., QSY quencher) for quenching the fluorescent or other detectable label (e.g., VIC dye), and another quencher (e.g., QSY quencher) for quenching the other fluorescent or detectable label (e.g., FAM dye). A probe specific for the target viral nucleic acid is formed by the combination of the VIC dye and the QSY quencher. Likewise, a probe specific for the endogenous nucleic acid is formed by the combination of the FAM dye and the QSY quencher.

An assay kit for performing the first exemplary assay includes a PCR assay mixture, a RNA control, a control dilution buffer, and a master mix. The PCR assay mixture includes the forward and reverse primers specific to the target viral nucleic acid (e.g., the N/S SARS-CoV-2 gene targets) and an endogenous nucleic acid (e.g., RNase P gene target), for example, TaqCheck™ SARS-CoV-2 Fast PCR Assay (Thermo Fisher Scientific). The RNA control is a control that contains templates specific to the target viral nucleic acid and the endogenous nucleic acid, for example, TaqCheck™ SARS-CoV-2 Control (Thermo Fisher Scientific). The control dilution buffer is a buffer for diluting the RNA control, for example, the TaqCheck™ SARS-CoV-2 Control Dilution Buffer (Thermo Fisher Scientific). The master mix is, for example, TaqPath 1-Step RT-qPCR Master Mix, CG (Thermo Fisher Scientific).

Preparation of buffer-detergent mixture: A TBE-T mixture is prepared with TBE buffer (10×), Tween-20 detergent (10%) and nuclease-free water in a DNase and RNase-free tube according to the following Table A1:

TABLE A1

| | | Volume per | Volume per four |
|---|---|---|---|
| | Volume per | 96-well | 96-well |
| | well | plate^{[1]} | plates^{[1]} |
| TBE Buffer (10X)^{[2]} | 20 μL | 2.4 mL | 9.6 mL |
| Tween ®-20 Detergent | 10 μL | 1.2 mL | 4.8 mL |
| (10%)^{[3]} | | | |
| Nuclease-free water | 70 μL | 8.4 mL | 33.6 mL |
| Total volume | 100 μL | 12.0 mL | 48.0 mL |

^{[1]}Includes 25% overage.
^{[2]}The TBE Buffer has a final concentration of 2X in the TBE-T mix.
^{[3]}The Tween ®-20 Detergent has a final concentration of 1% by the TBE-T mix.

The TBE-T mixture (100 μL) is added to each well of a plate.

Preparation of sample: Samples (e.g., saliva sample) are heated at 95° C. for 30 minutes, and then allowed to equilibrate at room temperature. Each heat-treated sample (100 μL) is transferred to designated wells of the plate.

Preparation of RT-PCR reactions: A working stock of the RNA control is prepared by a two-step dilution. Step (a) includes pipetting 95.0 μL of control dilution buffer into a microcentrifuge tube, and then adding 5.0 μL of the RNA control. Step (b) includes adding 95.0 μL of the control dilution buffer into a second microcentrifuge tube, and then adding 5.0 μL of the dilution created in step (a).

Preparation of reaction mixture: A reaction mixture is prepared by combining the master mix, the PCR assay mixture, nuclease-free water in quantities sufficient for the number of RNA samples, one positive control and one ‘no template’ control. For instance, Table A2 is used for a 96-well plate.

TABLE A2

| | Volume per | Volume for n samples | Volume for 94 samples |
|---|---|---|---|
| Component | sample or control | plus 2 controls^{[1]} | plus 2 controls^{[1]} |
| Master Mix (4X) | 2.5 μL | 2.75 × (n + 2) μL | 264.0 μL |
| PCR Assay | 0.5 μL | 0.55 × (n + 2) μL | 52.8 μL |
| mixture | | | |
| Nuclease-free | 2.0 μL | 2.2 × (n + 2) μL | 211.2 μL |
| water | | | |
| Total Reaction | 5.0 μL | — | 528.0 μL |
| Mix volume | | | |

^{[1]}All volumes include 10% overage for pipette error.

A reaction plate is set up according to Table A3.

TABLE A3

| | Volume per | | |
|---|---|---|---|
| | reaction | | |
| | | Positive Control | No Template Control |
| Component | Sample reaction | reaction | reaction |
| Reaction Mixture | 5.0 μL | 5.0 μL | 5.0 μL |
| Prepared sample | 5.0 μL | — | — |
| (saliva + TBE-T) | | | |
| Positive Control | — | 2.0 μL | — |
| (diluted Control from | | | |
| step 3) | | | |
| Nuclease-free water | — | 3.0 μL | 5.0 μL |
| Total volume | 10.0 μL | 10.0 μL | 10.0 μL |

PCR settings and Thermal Protocol: The analysis type is a standard curve, the run mode is fast, and the passive reference is ROX. FAM is set up as a reporter dye for the endogenous nucleic acid (e.g., RNase P), and VIC is set up as reporter dye for the target viral nucleic acid.

The thermal protocol is set up, and ran, according to the instrument being used (for example, see Tables A4 and A5).

TABLE A4
7500 Fast Real-time PCR Instrument

| | | | | | Number of |
|---|---|---|---|---|---|
| Step | Temperature | Ramp rate | Time | | cycles |
| Reverse | 50° C. | 100% | 4 | minutes | 1 |
| transcription | | | | | |
| Activation | 95° C. | 100% | 2 | minutes | 1 |
| Denaturation | 95° C. | 100% | 1 | second | 40 |
| Anneal/ | 60° C. | 100% | 24 | seconds | |
| extension | | | | | |

TABLE A5
QuantStudio ™ 5 Real-time PCR
Instrument, 96-well, 0.2-mL block

| | | | | | Number of |
|---|---|---|---|---|---|
| Step | Temperature | Ramp rate | Time | | cycles |
| Reverse | 50° C. | 3.49° C. per | 4 | minutes | 1 |
| transcription | | second | | | |
| Activation | 95° C. | 3.49° C. per | 2 | minutes | 1 |
| | | second | | | |
| Denaturation | 95° C. | 3.49° C. per | 1 | second | 40 |
| | | second | | | |
| Anneal/ | 60° C. | 2.7° C. per | 20 | seconds | |
| extension | | second | | | |

### Exemplary Assay 2

A second exemplary assay is a multiplex real-time RT-PCR assay for the detection of viral RNA in samples. The multiplex real-time RT-PCR assay can be for the detection of RNA from SARS-CoV-2 in upper respiratory samples (such as nasopharyngeal, oropharyngeal, nasal, and mid-turbinate swabs, and nasopharyngeal aspirate) and bronchoalveolar lavage (BAL) samples from individuals suspected of COVID-19, for example, the TaqPath™ COVID-19 Combo Kit (Thermo Fisher Scientific).

The second exemplary assay comprises forward and reverse primers specific to a different target genomic regions (e.g., ORF1ab, N gene, S gene, MS2 gene targets), and an endogenous nucleic acid (e.g., bacteriophage MS2). The second exemplary assay further comprises a fluorescent or other detectable label (e.g., FAM dye) for detecting a first target nucleic acid (e.g., ORF1ab), a fluorescent or other detectable label (e.g., VIC dye) for detecting a second target nucleic acid (e.g., N gene), a fluorescent or other detectable label (e.g., ABY dye) for detecting a third target nucleic acid (e.g., S gene), and a fluorescent or other detectable label (e.g., JUN dye) for detecting the endogenous nucleic acid (e.g., bacteriophage MS2). The second exemplary assay may also comprise one or more quenchers (e.g., QSY quenchers) for quenching the fluorescent or other detectable labels.

An assay kit for performing the second exemplary assay includes a PCR assay multiplex mixture, a RNA control, and a control dilution buffer. The PCR assay multiplex mixture includes the forward and reverse primers specific to the target nucleic acids (e.g., ORF1ab, N gene, S gene, MS2 gene targets), and the endogenous nucleic acid (e.g., bacteriophage MS2), for example, TaqPath™ COVID-19 RT-PCR Kit (Thermo Fisher Scientific). The RNA control is a control that contains templates specific to the target viral nucleic acid and the endogenous nucleic acid, for example, TaqPath™ COVID-19 Control (Thermo Fisher Scientific). The control dilution buffer is a buffer for diluting the RNA control, for example, the TaqPath™ COVID-19 Control Dilution Buffer (Thermo Fisher Scientific). The master mix is, for example, TaqPath 1-Step Multiplex Master Mix (No ROX) (Thermo Fisher Scientific).

RNA Extraction: RNA can be extracted using an automated method (as detailed below), or manually. Manual RNA extraction can be performed from a sample input volume of 200 μL or using either the MagMAX™ Viral/Pathogen Nucleic Acid Isolation Kit (Thermo Fisher Scientific) or the MagMAX™ Viral/Pathogen II Nucleic Acid Isolation Kit (Thermo Fisher Scientific).

Preparation of Processing Plates: Prepare processing plates according to Table B1.

TABLE B1

| | | | | Volume | |
|---|---|---|---|---|---|
| | Plate | | | per | |
| Plate ID | position | Plate type | Reagent | well | |
| Wash 1 | 2 | KingFisher ™ | Wash | 500 | μL |
| Plate | | Deep-Well | Solution | | |
| Wash 2 | 3 | 96 Plate | 80% | 1,000 | μL |
| Plate | | | Ethanol | | |
| Elution | 4 | | Elution | 50 | μL |
| Plate | | | Solution | | |

| | | |
|---|---|---|
| Tip Comb | 5 | Place a KingFisher ™ 96 tip comb for |
| Plate | | DW magnets in a KingFisher ™ 96 |
| | | KF microplate |

Preparation of Binding Bead Mix: Prepare Binding Bead Mix according to Table B2.

TABLE B2

| Component | Volume per well^{[1]} | |
|---|---|---|
| Binding Solution | 265 | μL |
| Total Nucleic Acid Magnetic Beads | 10 | μL |
| Total volume per well | 275 | μL |

^{[1]}Include 10% overage when making the Binding Bead Mix for use with multiple reactions.

reactions.

Preparation of Sample Plate: Proteinase K (5 μL) is added to each well of the 96-well plate.

The sample (200 μL) is added to each sample well.

Nuclease-free water (not DEPCT-treated) (200 μL) is added to the negative control well.

Binding Bead Mix (275 μL) is added to sample well, and the negative control well.

The MS2 Phage control (5 μL) is added to each sample well and the negative control well.

Process Samples: The samples are processed on a magnetic particle processor, for example, KingFisher™ Flex Magnetic Particle Processor. The samples are eluted in 50 μL of Elution Solution.

Preparation of RT-PCR Reactions: A working stock of the RNA control is prepared by a two-step dilution. Step (a) includes pipetting 98.0 μL of control dilution buffer into a microcentrifuge tube, and then adding 2.0 μL of the RNA control. Step (b) includes adding 87.5 μL of the control dilution buffer into a second microcentrifuge tube, and then adding 12.5 μL of the dilution created in step (a).

Preparation of reaction mixture: A reaction mixture is made by combining the master mix, the PCR assay mixture, and nuclease-free water in quantities sufficient for the number of RNA samples, one positive control and one negative control. For instance, Table B3 is used for a 96-well plate.

TABLE B3

| | | Volume | Volume |
|---|---|---|---|
| | Volume | for n RNA | for 94 RNA |
| | per RNA | samples | Samples |
| | sample or | plus 2 | plus 2 |
| Component | control | controls | controls |
| Master Mix | 6.25 μL | 6.875 × | 660 μL |
| (No ROX ™) (4X) | | (n + 2) μL | |
| PCR Assay | 1.25 μL | 1.375 × | 132 μL |
| Multiplex | | (n + 2) μL | |
| mixture | | | |
| Nuclease-free | 7.50 μL | 8.25 × | 792 μL |
| Water | | (n + 2) μL | |
| Total Reaction | 15.0 μL | — | 1584 μL |
| Mix volume | | | |

A reaction plate is set up according to Table B4.

TABLE B4

| | Volume per reaction | | |
|---|---|---|---|
| | RNA | Positive | Negative |
| | Sample | Control | Control |
| Component | reaction | reaction | reaction |
| Reaction Mix | 15.0 μL | 15.0 μL | 15.0 μL |
| Purified sample RNA | 10.0 μL | — | — |
| (from RNA extraction) | | | |
| Positive Control | — | 2.0 μL | — |
| (diluted Control) | | | |
| Nuclease-free Water | — | 8.0 μL | — |
| Purified Negative | — | — | 10.0 μL |
| Control (from RNA | | | |
| extraction) | | | |
| Total volume | 25.0 μL | 25.0 μL | 25.0 μL |

PCR settings and Thermal Protocol: The analysis type is a standard curve, the run mode is standard, and passive reference is none. JUN is set up as a reporter dye for the endogenous nucleic acid (e.g., MS2), FAM is set up as a reporter dye for a first target nucleic acid (e.g., ORF1ab), VIC is set up as a reporter dye for a second target viral nucleic acid (e.g., N gene), ABY is set up as a reporter dye for a third target viral nucleic acid (e.g., S gene).

The thermal protocol is set up, and ran, according to the instrument being used (for example, see Table B5).

TABLE B5

| | | | | Number |
|---|---|---|---|---|
| Step | Temperature | Time | | of cycles |
| UNG incubation | 25° C. | 2 | minutes | 1 |
| Reverse transcription | 53° C. | 10 | minutes | 1 |
| Activation | 95° C. | 2 | minutes | 1 |
| Denaturation | 95° C. | 3 | seconds | 40 |
| Anneal/extension | 60° C. | 30 | seconds | |

### Exemplary Assay 3

A third exemplary assay is a multiplex real-time RT-PCR assay for the detection of viral RNA in samples. The multiplex real-time RT-PCR assay can be for the detection of RNA from SARS-CoV-2 in human samples, for example, the TaqMan™ SARS-CoV-2 with RNase P Assay 2.0 (Thermo Fisher Scientific).

The third exemplary assay comprises forward and reverse primers specific to a different target genomic regions (e.g., ORF1a, N gene, S gene, RNase P gene targets), and an endogenous nucleic acid (e.g., RNase P). The third exemplary assay further comprises a fluorescent or other detectable label (e.g., FAM dye) for detecting a first target nucleic acid (e.g., ORF1a), a fluorescent or other detectable label (e.g., VIC dye) for detecting a second target nucleic acid (e.g., N gene), a fluorescent or other detectable label (e.g., ABY dye) for detecting a third target nucleic acid (e.g., S gene), and a fluorescent or other detectable label (e.g., JUN dye) for detecting the endogenous nucleic acid (e.g., RNase P). The third exemplary assay also comprises quenchers (e.g., QSY quenchers) for quenching the fluorescent or other detectable labels. The quenchers in the third exemplary assay do not fluoresce.

An assay kit for performing the third exemplary assay includes a PCR assay multiplex mixture, a RNA control, and a control dilution buffer. The PCR assay multiplex mixture includes the forward and reverse primers specific to the target nucleic acids (e.g., ORF1a, N gene, and ORF1b gene targets), and the endogenous nucleic acid (e.g., RNase P), for example, TaqMan™ SARS-CoV-2 with RNase P Assay 2.0 (Thermo Fisher Scientific). The RNA control is a control that contains templates specific to the target viral nucleic acid and the endogenous nucleic acid, for example, TaqMan™ SARS-CoV-2 Plus Control (Thermo Fisher Scientific). The control dilution buffer is a buffer for diluting the RNA control, for example, the TaqMan™ Control Dilution Buffer (Thermo Fisher Scientific). The master mix is, for example, TaqPath 1-Step Multiplex Master Mix (No ROX) (Thermo Fisher Scientific).

### RNA Extraction

Preparation of Processing Plates: Processing plates are prepared according to Table C1.

TABLE C1

| | Plate | | | Volume | |
|---|---|---|---|---|---|
| Plate ID | position | Plate type | Reagent | per well | |
| Wash 1 Plate | 2 | KingFisher ™ 96 | Wash Solution | 500 | μL |
| Wash 2 Plate | 3 | Deep-Well Plate | 80% Ethanol | 1,000 | μL |
| Elution Plate | 4 | | Elution Solution | 50 | μL |

| | | |
|---|---|---|
| Tip Comb | 5 | Place a KingFisher ™ 96 |
| Plate | | tip comb for DW magnets in a |
| | | KingFisher ™ 96 KF microplate |

Preparation of Binding Bead Mix: Binding Bead Mix is prepared according to Table C2.

TABLE C2

| | | |
|---|---|---|
| | Volume | Volume per |
| Component | per well^{[1]} | 96-well plate |

| | | | | |
|---|---|---|---|---|
| Binding Solution | 265 | μL | 28.0 | mL |
| Total Nucleic Acid | 10 | μL | 1.1 | mL |
| Magnetic Beads | | | | |
| Total volume | 275 | μL | 29.1 | mL |

^{[1]}Include 10% overage when preparing the Binding Bead Mix for use with multiple reactions.

Preparation of Sample Plate: Binding Bead Mix (275 μL) is added to sample well, and the negative control well. The sample (200 μL) is added to each sample well.

Nuclease-free water (not DEPCT-treated) (200 μL) is added to the negative control well.

Proteinase K (5 μL) is added to each well of the 96-well plate.

Process Samples: The samples are processed on a magnetic particle processor, for example, KingFisher Flex Magnetic Particle Processor. The samples are eluted in 50 μL of Elution Solution.

Preparation of RT-PCR Reactions: A working stock of the RNA control is prepared by a two-step dilution. Step (a) includes pipetting 100.0 μL of control dilution buffer into a microcentrifuge tube, and then adding 2.0 μL of the RNA control. Step (b) includes adding 110.0 μL of the control dilution buffer into a second microcentrifuge tube, and then adding 2.0 μL of the dilution created in step (a).

Preparation of reaction mixture: A reaction mixture is made by combining the master mix, and the PCR assay mixture in quantities sufficient for the number of RNA samples, one positive control and one negative control. For instance, Table C3 is used for a 96-well plate.

TABLE C3

| | | | Volume | Volume |
|---|---|---|---|---|
| | Volume | | for n RNA | for 94 RNA |
| | per RNA | | Samples | Samples |
| | Sample or | | plus 2 | plus 2 |
| Component | Control | | Controls | Controls |
| Master Mix | 6.25 | μL | 6.875 × | 660 μL |
| (No ROX ™) (4X) | | | (n + 2) μL | |
| RNase P Assay | 1.25 | μL | 1.375 × | 132 μL |
| mixture | | | (n + 2) μL | |
| Total Reaction | 7.5 | μL | — | 792 μL |
| Mix volume | | | | |

A reaction plate is set up according to Table C4.

TABLE C4

| Volume per reaction | | | |
|---|---|---|---|
| | RNA | Positive | Negative |
| | Sample | Control | Control |
| | reaction | reaction | reaction |

| | | | | | | |
|---|---|---|---|---|---|---|
| Reaction Mix | 7.5 | μL | 7.5 | μL | 7.5 | μL |
| (from step 3) | | | | | | |

| | | | | |
|---|---|---|---|---|
| Purified sample RNA | 17.5 | μL | — | — |
| (from RNA extraction) | | | | |

| | | | | |
|---|---|---|---|---|
| Positive Control | — | 17.5 | μL | — |
| (diluted Control) | | | | |

| | | | | |
|---|---|---|---|---|
| Negative Control | — | — | 17.5 | μL |
| (from RNA extraction) | | | | |

| | | | | | | |
|---|---|---|---|---|---|---|
| Total volume | 25.0 | μL | 25.0 | μL | 25.0 | μL |

PCR settings and Thermal Protocol: The analysis type is a standard curve, the run mode is standard. JUN is set up as a reporter dye for the endogenous nucleic acid (e.g., RNase P), FAM is set up as a reporter dye for a first target nucleic acid (e.g., ORF1a), VIC is set up as a reporter dye for a second target viral nucleic acid (e.g., N gene), ABY is set up as a reporter dye for a third target viral nucleic acid (e.g., ORF1b).

The thermal protocol is set up, and ran, according to the instrument being used (for example, see Table C5).

TABLE C5

| | | | | | Number |
|---|---|---|---|---|---|
| Step | Temperature | Ramp rate | Time | | of cycles |
| | | 1.6° C. | 2 | minutes | 1 |
| Reverse | 53° C. | per second | 10 | minutes | 1 |
| transcription | | | | | |
| Preincubation | 85° C. | | 10 | minutes | 1 |
| Activation | 95° C. | | 2 | minutes | 1 |
| Denaturation | 95° C. | | 3 | seconds | 40 |
| Anneal/extension | 60° C. | | 30 | seconds | |

### Exemplary Assay 4

A fourth exemplary assay is a multiplex real-time RT-PCR assay for the detection of viral RNA in samples. The multiplex real-time RT-PCR assay can be for the detection of RNA from SARS-CoV-2 in raw human saliva samples, for example, the TaqMan™ SARS-CoV-2 Fast PCR Combo Kit 2.0 (Thermo Fisher Scientific).

The fourth exemplary assay comprises forward and reverse primers specific to a different target genomic regions (e.g., ORF1a, N gene, ORF1b gene targets), and an endogenous nucleic acid (e.g., RNase P). The fourth exemplary assay further comprises a fluorescent or other detectable label (e.g., FAM dye) for detecting a first target nucleic acid (e.g., ORF1a), a fluorescent or other detectable label (e.g., VIC dye) for detecting a second target nucleic acid (e.g., N gene), a fluorescent or other detectable label (e.g., ABY dye) for detecting a third target nucleic acid (e.g., ORF1b gene), and a fluorescent or other detectable label (e.g., JUN dye) for detecting the endogenous nucleic acid (e.g., RNase P). The fourth exemplary assay also comprises quenchers (e.g., QSY quenchers) for quenching the fluorescent or other detectable labels. The quenchers in the fourth exemplary assay do not fluoresce.

An assay kit for performing the fourth exemplary assay includes a PCR assay multiplex mixture, a RNA control, a lysis buffer and a control dilution buffer. The PCR assay multiplex mixture includes the forward and reverse primers specific to the target nucleic acids (e.g., ORF1a, N gene, and ORF1b gene targets), and the endogenous nucleic acid (e.g., RNase P), for example, TaqMan™ SARS-CoV-2 FAST PCR Assay 2.0 (Thermo Fisher Scientific). The RNA control is a control that contains templates specific to the target viral nucleic acid and the endogenous nucleic acid, for example, TaqMan™ SARS-CoV-2 Plus Control (Thermo Fisher Scientific). The control dilution buffer is a buffer for diluting the RNA control, for example, the TaqMan™ SARS-CoV-2 Control Dilution Buffer (Thermo Fisher Scientific). The lysis buffer is, for example, SalivaReady™ Solution (Thermo Fisher Scientific). The master mix is, for example, TaqPath 1-Step Multiplex Master Mix (No ROX) (Thermo Fisher Scientific).

Preparation of Samples: Lysis solution (20 μL) is added to each well of a 96-well plate at room temperature, and then test samples (20 μL) are added to each well containing the lysis solution.

The plate is heated in a thermal cycler using the thermal conditions shown in Table D1.

TABLE D1

| | | | Number |
|---|---|---|---|
| Step | Temperature | Time | of cycles |
| 1 | 62° C. | 5 minutes | 1 |
| 2 | 92° C. | 5 minutes | 1 |
| 3 | 4° C. | Hold^{[1]} | 1 |

^{[1]}The thermal cycler can be stopped once at 4° C.

Preparation of RT-PCR Reactions: A working stock of the RNA control is prepared by a two-step dilution. Step (a) includes pipetting 120.0 μL of control dilution buffer into a microcentrifuge tube, and then adding 2.0 μL of the RNA control. Step (b) includes adding 120.0 μL of the control dilution buffer into a second microcentrifuge tube, and then adding 2.0 μL of the dilution created in step (a).

Preparation of reaction mixture: A reaction mixture is made by combining the master mix, and the PCR assay mixture in quantities sufficient for the number of RNA samples, one positive control and one negative control. For instance, Table D2 is used for a 96-well plate.

TABLE D2

| | | Volume | Volume |
|---|---|---|---|
| | | for n | for 94 |
| | Volume per | samples | samples |
| | sample or | plus 2 | plus 2 |
| Component | control | controls^{[1]} | controls^{[1]} |
| Master Mix (No ROX ™) (4X) | 5.0 μL | 5.5 × | 528.0 μL |
| | | (n + 2) μL | |
| PCR Assay mixture (20X) | 1.0 μL | 1.1 × | 105.6 μL |
| | | (n + 2) μL | |
| Total Reaction Mix volume | 6.0 μL | — | 633.6 μL |

^{[1]}All volumes include 10% overage for pipette error.

A reaction plate is set up according to Table D3.

TABLE D3

| | Volume per reaction | | |
|---|---|---|---|
| | | Positive | Negative |
| | Sample | Control | Control |
| Component | reaction | reaction | reaction |
| Reaction Mix | 6.0 μL | 6.0 μL | 6.0 μL |
| Prepared sample | 14.0 μL | — | — |
| (sample plus | | | |
| lysis solution) | | | |
| Positive Control | — | 14.0 μL | — |
| (diluted Control) | | | |
| Nuclease-free water | — | — | 14.0 μL |
| Total volume | 20.0 μL | 20.0 μL | 20.0 μL |

PCR settings and Thermal Protocol: The analysis type is a standard curve, the run mode is standard, and the passive reference is none. JUN is set up as a reporter dye for the endogenous nucleic acid (e.g., RNase P), FAM is set up as a reporter dye for a first target nucleic acid (e.g., ORF1a), VIC is set up as a reporter dye for a second target viral nucleic acid (e.g., N gene), ABY is set up as a reporter dye for a third target viral nucleic acid (e.g., ORF1b).

The thermal protocol is set up, and ran, according to the instrument being used (for example, see Table D4).

TABLE D4
QuantStudio ™ 5 Real-time PCR
Instrument, 96-well, 0.2-mL block

| | | | | | Number |
|---|---|---|---|---|---|
| Step | Temperature | Ramp rate | Time | | of cycles |
| Reverse | 53° C. | 3.2° C. | 5 | minutes | 1 |
| transcription | | per second | | | |
| Preincubation | 85° C. | 3.2° C. | 5 | minutes | 1 |
| | | per second | | | |
| Activation | 95° C. | 3.2° C. | 2 | minutes | 1 |
| | | per second | | | |
| Denaturation | 95° C. | 3.2° C. | 1 | second | 40 |
| | | per second | | | |
| Anneal/extension | 62° C. | 2.5° C. | 30 | seconds | |
| | | per second | | | |

While the exemplary assays are described using a 96-well reaction plate, embodiments are not limited thereto, and the exemplary assays may be optimized for performance on an instrumentation that includes more than 96 wells (e.g., QuantStudio™ 5 Real-time PCR Instrument, 384-well block, and QuantStudio™ 7 Flex Real-time PCR Instrument, 384-well block).

The TaqCheck™ SARS-CoV-2 Fast PCR Assay User Guide (Rev. B or higher), the TaqPath™ COVID-19 Combo Kit and TaqPath™ COVID-19 Combo Kit Advanced Instructions for Use (Rev. J or higher), the TaqMan™ SARS-CoV-2 with RNase P Assay 2.0 User Guide (Rev. B or higher), and the TaqMan™ SARS-CoV-2 Fast PCR Combo Kit 2.0 User Guide (Rev. A or higher), are herein incorporated by reference.

As described in more detail elsewhere herein, certain assays may perform better than others. For example, preferred embodiments minimize the amount of crosstalk between dye channels, particularly crosstalk that effects an endogenous control such as RNase P.

### Methods for Quantifying Target Nucleic Acid Across Multiple Samples

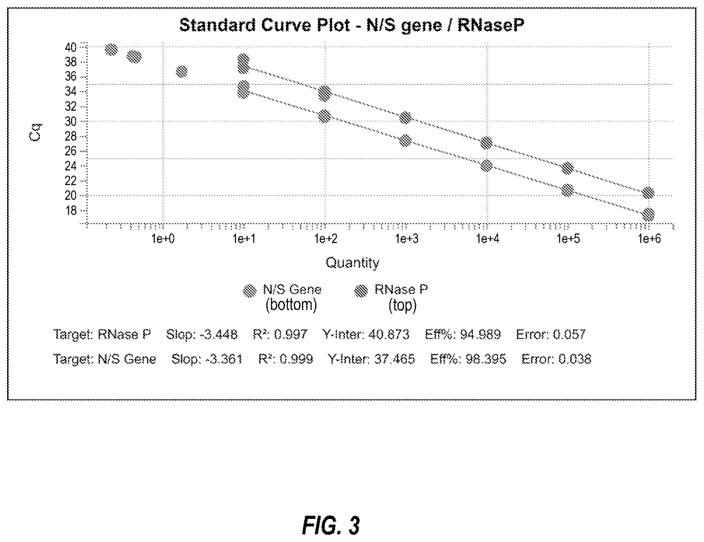

FIG. 2 illustrates an exemplary method 100 for quantifying a target nucleic acid and enabling comparison of nucleic acid levels between multiple samples. The method 100 includes the step of providing two or more test samples (step 102). Each of the test samples include, or are suspected as including, the target nucleic acid. As disclosed herein, the target nucleic acid may be viral, bacterial, fungal, or eukaryotic. The target nucleic acid may be from a pathogen. The target nucleic acid may be from a respiratory pathogen. In some embodiments, the target nucleic acid is from SARS-CoV-2. Some embodiments quantify a single target nucleic acid (e.g., a singleplex reaction), whereas other embodiments quantify multiple target nucleic acids (e.g., a multiplex reaction). In some embodiments, the target nucleic acid may be artificial, synthetic, or foreign nucleic acid.

In some embodiments, the two or more test samples are derived from the same subject. In some embodiments, the two or more test samples are derived from the same location of the same subject (e.g., from the same or different nostril such as from the right nostril, the left nostril, or both nostrils). Other embodiments may quantify and compare samples obtained from different subjects, or may quantify and compare samples obtained from the same subject at different locations, but there are particular benefits associated with comparing samples obtained at different time periods from the same subject to enable the monitoring of progression of target nucleic acid levels. Samples may be obtained at different time periods separated by any appropriate time period. For example, sample collection may be separated by a time period of 1 hour, 2 hours, 3 hours, 6 hours, 12 hours, 24 hours, 48 hours, 72 hours, 96 hours, one week, two weeks, three weeks, four weeks, six weeks, eight weeks, ten weeks, three months, four months, five months, six months, or a time period range with endpoints defined by any two of the foregoing values. Time between sample collection may depend on the particular nucleic acid target, expected pathogenicity, expected incubation period, expected infection period, and the like.

In some embodiments, the assay may collect samples from at least one of three sites using nasal swabs—the right nostril, the left nostril, and both nostrils. In some embodiments, the swab samples are collected from the same nostril or from the different nostril. In some embodiments, the assay may use one or more collection protocol(s) for nasal swabs as specified below:

### Right Nostril Sample

a. Remove the cap of the sample collection tube
   i. Keep the cap and collection tube near the patient
b. Open the swab package
   i. Starting from the handle end of the swab package, peel open the paper backing. Pull swab out of its packaging by the handle
   ii. Do not touch the soft tip with hands or lay it down on any surface
c. Rotate the swab tip in the right nostril, 4 times
   i. Insert the soft tip into the right nostril until it is no longer visible. Using medium pressure against the inside of your nostril the whole time, rub the swab in a circular motion at least 4 times.
   ii. Swab should not go further than ½ inch.
d. Put the swab into the collection tube
   i. The soft tip of the swab that went into the nose should go into the tube first
e. Press the cap of the collection tube firmly closed
f. Put the collection tube in the biohazard bag.

### Left Nostril Sample

a. Remove the cap of the sample collection tube
   i. Keep the cap and collection tube near the patient
b. Open the swab package
   i. Starting from the handle end of the swab package, peel open the paper backing. Pull swab out of its packaging by the handle
   ii. Do not touch the soft tip with hands or lay it down on any surface
c. Rotate the swab tip in the left nostril, 4 times
   i. Insert the soft tip into the left nostril until it is no longer visible. Using medium pressure against the inside of your nostril the whole time, rub the swab in a circular motion at least 4 times.
   ii. Swab should not go further than ½ inch.
d. Put the swab into the collection tube
   i. The soft tip of the swab that went into the nose should go into the tube first
e. Press the cap of the collection tube firmly closed
f. Put the collection tube in the biohazard bag.

### Both Nostrils Sample

a. Remove the cap of the sample collection tube
   i. Keep the cap and collection tube near the patient
b. Open the swab package
   i. Starting from the handle end of the swab package, peel open the paper backing. Pull swab out of its packaging by the handle
   ii. Do not touch the soft tip with hands or lay it down on any surface
c. Rotate the swab tip in the right nostril, 4 times
   i. Insert the soft tip into the right nostril until it is no longer visible. Using medium pressure against the inside of your nostril the whole time, rub the swab in a circular motion at least 4 times.
   ii. Swab should not go further than ½ inch.
d. Rotate the swab tip in the left nostril, 4 times
   i. Insert the soft tip into the left nostril until it is no longer visible. Using medium pressure against the inside of your nostril the whole time, rub the swab in a circular motion at least 4 times.
e. Put the swab into the collection tube
   i. The soft tip of the swab that went into the nose should go into the tube first
f. Press the cap of the collection tube firmly closed
g. Put the collection tube in the biohazard bag.
h. Seal the biohazard bag

The test samples may be collected from any suitable source. In some embodiments, the test samples are not blood samples. In some embodiments, the test samples are saliva samples. In some embodiments, the test samples are swab samples. As used herein, the terms “swab sample”, “swab-based sample”, and similar terms refer to samples that include a swab itself and to fluid and/or cellular biomass samples obtained from swabs. The terms include samples where the swab itself is directly subjected to a subsequent extraction and/or quantification process, as well as where the one or more intervening process steps are utilized before extraction, amplification, and/or quantification of the target nucleic acid. As described elsewhere herein, swab samples may be obtained from any anatomical location associated with the target nucleic acid. For respiratory pathogens, oropharyngeal or nasopharyngeal swabs are typical.

The method 100 also includes the step of providing a set of control samples each having a known concentration of a control nucleic acid (step 104). The control samples may include, for example, serial dilutions of a known quantity of the control nucleic acid. In some embodiments, the control nucleic acids are substantially the same as or include the target nucleic acid. For example, the control nucleic acids may be prepared by amplifying the target nucleic acids to obtain high copy number concentrations, and then performing the serial dilutions to form the control samples. In some embodiments, the control nucleic acids comprise whole or partial genomes of the target microorganism or virus. In some embodiments, the method may generate a standard curve to report out viral load values in IU/mL and/or copies/mL.

The method 100 also includes the step of amplifying at least a portion of the target nucleic acid in each of the test samples by subjecting each test sample to amplification conditions in the presence of target-specific primers (step 106) and the step of amplifying at least a portion of the control nucleic acid in each of the control samples by subjecting each control sample to amplification conditions in the presence of control primers (step 108). These steps may be performed simultaneously or sequentially in any order. In some embodiments, amplification is carried out via PCR. In some embodiments, amplification of the target nucleic acid and/or the control nucleic acid comprises a reverse transcription reaction. Additional details regarding amplification are provided elsewhere herein, and it will be understood that those details are applicable to the method 100.

In some embodiments, the control primers and the target-specific primers are the same. In some embodiments, the control nucleic acids are capable of being amplified with the target-specific primers, even though the control nucleic acids and the target nucleic acids need not necessarily be the same. In some embodiments, the target nucleic acid is a SARS-CoV-2 nucleic acid, and the target-specific primers are specific to one or more of the Orf1a gene, the Orf1b gene, the N gene, or the S gene of SARS-CoV-2.

The illustrated method 100 also includes the step of generating a standard curve using the results of amplifying the control nucleic acids (step 110) and determining an absolute quantity (AQ) of target nucleic acid in each test sample using the standard curve (112). The standard curve may be utilized as known in the art to provide the absolute quantity. For example, the absolute quantity may be determined via mathematical extrapolation from one or more control sequence standard or interpolation between two or more control sequence standards, or by reference to a stored (e.g., digital copy) of a standard curve. As discussed above, the “absolute quantity” or “AQ” of each separate test sample is affected by the amount of organic matter in the sample, which varies from sample to sample, particularly for swab-based samples.

The method 100 also includes the step of amplifying an endogenous nucleic acid in each of the test samples by subjecting each test sample to amplification conditions in the presence of endogenous sequence primers (step 114). This step may be performed before, after, or during steps 106 and 108. The endogenous nucleic acid is present in the organic matter (e.g., cells and/or extracellular material such as mucous or cellular debris) of the test sample and is therefore expected to be present in amounts proportional to the amount of organic matter in the sample. That is, detection of the target and the endogenous control may be impacted by amount of biomass of specimen. For example, under-collection of biomass may result from insufficient sampling, whereas when the target is present the collected biomass may carry proportional amounts of infectious agent and endogenous control.

Preferable endogenous nucleic acids are stably expressed across test samples and minimally affected by test conditions, extraction processes, and subject differences. Some embodiments utilize protein-coding endogenous nucleic acids such as beta-actin or glyceraldehyde 3-phosphate dehydrogenase (GAPDH). More preferred embodiments typically utilize sequences that code for ribosomal RNA molecules rather than proteins. For example, in some preferred embodiments, the endogenous nucleic acid is an RNase P sequence. Other examples include: 18S ribosomal RNA; peptidylprolyl isomerase A (cyclophilin A); ribosomal protein L13a; ribosomal protein large P0; beta-2-microglobulin; tyrosine 3-monooxygenase/tryptophan 5-monooxygenase activation protein, zeta polypeptide; succinate dehydrogenase; transferrin receptor (p90, CD71); aminolevulinate, delta-, synthase 1; glucuronidase, beta; hydroxymethyl-bilane synthase; hypoxanthine phosphoribosyltransferase 1; TATA box binding protein; and tubulin, beta polypeptide.

The method 100 also includes the step of determining a correction factor (“RQ”) for each test sample based on relative levels of endogenous nucleic acid in each test sample (step 116). In some embodiments, the method includes establishing a baseline correction factor for a first test sample associated with a first time point and determining subsequent correction factors for subsequent test samples relative to the baseline correction factor. For example, the first correction factor for the first test sample from the first time point may be set at 1 (or some other appropriate value) based on a ratio of the measured target nucleic acid to the measured endogenous nucleic acid at the first time point. Subsequent correction values may be adjusted from the baseline based on different ratios of measured target nucleic acid to measured endogenous nucleic acid at the subsequent time points.

In some embodiments, the correction factor (RQ) for each nth sample may be calculated as:

$$R Q ( n ) = \frac{{2}^{- ( C q ( n ) - C q ( 0 ) ) target}}{{2}^{- ( C q ( n ) - C q ( 0 ) ) c ontrol}}$$

where (Cq(n)−Cq(0))_{target} is the difference between the measured Cq of the target nucleic acid at the nth sample and the Cq of the target nucleic acid at the initial “T₀” sample, and (Cq(n)−Cq(0))_{control} is the difference between the measured Cq of the endogenous control nucleic acid at the nth sample and the Cq of the control nucleic acid at the initial “T₀” sample. A mathematically equivalent version of the above formula is:

$$R Q ( n ) = \frac{{2}^{- ( C q ( target ) - C q ( control ) ) n}}{{2}^{- ( C q ( target ) - C q ( c ontrol ) ) 0}}$$

where (Cq(target)−Cq(control))ₙ is the difference between the measured Cq of the target nucleic acid and the Cq of the control nucleic acid for a given test sample at the nth time point, and (Cq(target)−Cq(control))ₙ is the difference between the measured Cq of the target nucleic acid and the Cq of the control nucleic acid for the initial “T₀” test sample.

In the above equations for RQ(n), the numeral 2 is used as the base in both the numerator and denominator. This is sufficient for most cases. In certain embodiments, the numeral 2 may be replaced by an amplification efficiency measurement for the target nucleic acid (numerator) and/or for the endogenous control nucleic acid (denominator). For example, the correction factor (RQ) for each nth sample may be calculated as:

$$R Q ( n ) = \frac{{( 1 + Eff . Target )}^{- ( C q ( n ) - C q ( 0 ) ) target}}{{( 1 + Eff . Control )}^{- ( C q ( n ) - C q ( 0 ) ) c ontrol}}$$

where “Eff. Target” is the PCR efficiency of the target nucleic acid, “Eff. Control” is the PCR efficiency of the control nucleic acid, (Cq(n)−Cq(0))_{target} is the difference between the measured Cq of the target nucleic acid at the nth sample and the Cq of the target nucleic acid at the initial “T₀” sample, and (Cq(n)−Cq(0))_{control} is the difference between the measured Cq of the endogenous control nucleic acid at the nth sample and the Cq of the control nucleic acid at the initial “T₀” sample.

The method also includes the step of determining a corrected quantity (i.e., “nQuant”, “corrected Quant”, or “corrected AQ”) of target nucleic acid in each test sample by normalizing the absolute quantity of target nucleic acid using the respective correction factors (step 118). In some embodiments, the corrected quantity of target nucleic acid, for each nth sample may be calculated as:

$$nQuant = {AQ}_{n} * R {Q}_{n}$$

The method 100 may also include the step of determining a corrected quantity of the subsequent test samples relative to the first test sample to illustrate relative change in target nucleic acid load of the test samples over time.

In some embodiments, the method may use a 1-gene, 2-gene, 3-gene, or 4-gene qPCR assay for the detection of SARS-CoV-2 in upper respiratory tract samples and an additional channel for RNaseP to ensure sample adequacy and for normalization. In some embodiments, the 1-gene, 2-gene, 3-gene or 4-gene are selected among Orf1a gene, the Orf1b gene, the N gene, or the S gene of SARS-CoV-2.

In preferred embodiments, the amplification efficiencies of the target nucleic acid and the endogenous nucleic acid are substantially similar. Otherwise, the ability to use their quantitation ratio as a useful correction factor degrades. In some embodiments, amplification of the control nucleic acids (which correspond to known amounts of the target nucleic acid) in the control samples (to generate the standard curve) and amplification of the endogenous nucleic acid have efficiencies that differ by no more than about 10%, no more than about 8%, no more than about 6%, or no more than about 4%. In some embodiments, an efficiency plot of amplification of the control nucleic acids in the control samples and an efficiency plot of amplification of the endogenous nucleic acids in the test samples have slopes (Cq/quantity) that differ by no more than about 10%, no more than about 8%, no more than about 6%, or no more than about 4%. Efficiency determinations typically inherently vary from 92-108% (e.g., about 80-130%, or about 85-120%).

### Sample Collection

In most instances, the sample is a swab sample. Examples include throat swabs (i.e., oropharyngeal swabs), nasal swabs (i.e., nasopharyngeal swabs), cheek swabs, saliva swabs, or other swabs, though it should be appreciated that SARS-CoV-2 or other targets may also be detected by analysis of other swab types and other sample types, such as urine samples, saliva samples, or other clinical samples. Such samples may be collected with a collection device such as a tube, a dish, a bag, a plate, or any other appropriate container. Though these types of samples may not vary as widely as swab samples, they still have the potential to vary in amount or concentration of organic matter from sample to sample, and the principles and benefits of the disclosed embodiments may therefore be beneficially applied to these types of samples as well.

The sample may be a veterinary sample, a clinical sample, a food sample, a forensic sample, an environmental sample (e.g., soil, dirt, garbage, sewage, air, or water), including food processing and manufacturing surfaces, or a biological sample. In some embodiments, the sample is a human sample. In some embodiments, the sample is a non-human sample. For instance, the sample may be from a non-human species such as a dog, cat, mink, livestock animals (e.g., pigs, cattle, sheep, goats), etcetera.

The sample can be collected by a healthcare professional in a healthcare setting, but in some instances, the sample may also be collected by the patient themselves or by an individual assisting the patient in self-collection. In some embodiments, the sample is a raw saliva sample collected—whether by self-collection or assisted/supervised collection—in a sterile tube or specifically-designed saliva collection device. The saliva collection tube/device may be a component of a self-collection kit having instructions for use, such as sample collection instructions, sample preparation or storage instructions, and/or shipping instructions. The raw saliva sample can be collected directly into a sealable container without any preservation solution or other fluid or substance in the container prior to receipt of the saliva sample within the container or as a result of closing/sealing the container.

In some embodiments, the nucleic acid fraction of the sample is extracted or purified from the sample—whether obtained via swab, from raw saliva, or other bodily fluid—prior to any detection of target nucleic acids therein. In some embodiments that utilize one or more swab-based samples, the associated swabs may be dried prior to extraction and/or amplification or may alternatively be stored in collection media. The collection media may comprise a liquid that preserves the swab during storage and/or during shipment between donor and testing site. The collection media is tested either directly or after extraction and purification of the nucleic acid target.

Alternatively, certain embodiments can be adapted to detect target nucleic acid directly from a raw sample without a specific nucleic acid purification and/or extraction step prior to its use in downstream detection assays (e.g., RT-qPCR). In some embodiments, the sample is pre-treated prior to use. This can include, for example, heating the sample, such as by placing the raw sample on a heat block/water bath set to a heating temperature (e.g., about 95° C.) for a pre-treatment period (e.g., about 30 minutes), followed by combining the sample with a buffer or lysis solution. The buffer or lysis solution can include, for example, any nucleic-acid-amenable buffer such as Tris-borate-EDTA (TBE) and may further include a detergent and/or emulsifier such as the polysorbate-type nonionic surfactant, Tween-20. The buffer or lysis solution may include a chaotropic agent and/or one or more enzymes, such as proteases, to help improve analyte detection by breaking down biological material and releasing analytes to make them more available for detection yet preserving nucleic acid targets.

A pre-heating step can provide many benefits, including, for example, breaking down mucus, making the sample more amenable to manipulation with laboratory equipment such as pipettes. The high heat can also cause thermal disruption of any prokaryotic and eukaryotic cells present in the sample and can also disrupt target organisms or virions present in the sample and thereby increase accessibility to any target nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of target nucleic acid within the sample via nucleic acid amplification reactions (e.g., PCR, RT-PCR, qPCR, RT-qPCR, or the like). The lysis solution can include a nucleic-acid-amenable buffer such as TBE (and/or suitable alternative known in the art) combined with a detergent and/or emulsifier such as Tween-20, the polysorbate-type nonionic surfactant (and/or suitable alternative known in the art). The detergent and/or emulsifier can promote better mixing of the reagents and may also act to increase accessibility to any target nucleic acid within the sample (e.g., by removing lipid envelopes from virions).

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the saliva/buffer mixture. In other embodiments, the sample is directly combined with a buffer/detergent mixture. As a non-limiting example, a set of patient samples can be prepared as compositions for downstream analysis and detection of viral sequence by adding a volume of heat-treated sample for each patient into one (or a plurality) of wells in a multi-well plate. A volume of a buffer/detergent mixture (e.g., TBE+Tween-20) can then be added to each well containing a patient sample. Alternatively, a multi-well plate can be loaded with a volume of a buffer/detergent mixture into which a volume of heat-treated saliva is added. Once combined, this probative template solution can be used immediately or stored for later analysis. Such probative template solutions can also be combined with PCR reagents (e.g., buffers, dNTPs, master mixes, etc.) prior to or after storage.

### Nucleic Acid Amplification & Detection

Amplified products (“amplicons”) resulting from use of one or more embodiments described herein can be generated, detected, and/or analyzed using any suitable method and on any suitable platform. In some embodiments, the nucleic acid targets may be single-stranded, double-stranded, or any other nucleic acid molecule of any size or conformation. The nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods described herein for detecting nucleic acid targets in a sample. In some embodiments, the PCR assays can be real time PCR or quantitative (qPCR) assays. In some embodiments, the PCR assays can be end point PCR assays.

In some embodiments, the primers described herein are used in nucleic acid assays at a concentration from about 100 nM to 1 mM (e.g., 300 nM, 400 nM, 500 nM, etc.), including intervening concentration amounts and ranges defined by endpoints selected from any two of the foregoing values. In some embodiments, probes described herein are also used in a nucleic acid assay and are provided at a concentration from about 50 nM to 500 nM (e.g., 75 nM, 125 nM, 250 nM, etc.), including intervening concentration amounts and ranges defined by endpoints selected from any two of the foregoing values.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels include but are not limited to 6FAM, ABY, VIC, JUN, FAM. Suitable quenchers include but are not limited to QSY (e.g., QSY7 and QSY21), BHQ (Black Hole Quencher) and DFQ (Dark Fluorescent Quencher).

In some multiplex assay embodiments, various genomic regions are detected. Where SARS-CoV-2 is a target, examples include assays configured to detect the Orf region (e.g., Orf1a, Orf1b, Orf1ab, Orf8), N protein region, S protein region, other genomic regions, and/or combinations thereof. Such multiplex assay embodiments may include multiple different probes for the same target genomic region in order to detect and/or distinguish between SARS-CoV-2 variants. For example, a multiplex assay that includes a target in the S Protein genomic region may include multiple different probes (each differentially labelled) for different variant forms of the targeted S Protein genomic region. Other target regions (including the N Protein and/or Orf regions) may also include multiple probes corresponding to different variant forms of such target regions. Optionally, in some embodiments, control sequence primers and/or probes (e.g., JUN-labeled probes), such as for amplification and/or detection of bacteriophage MS2 or human RNase P control sequences, are included in the multiplex assays using suitable primer/probe sequences (and may be included as singleplex assays as well).

Optionally, different qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4346907, 4306737, 4326659, 4316813, N8010560, 4309849, 4326270, 4343814 and 4343370). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR. For example, such applications include HER2/neu gene copy semi-quantitative detection in tissue sections, or RNA expression of analytes.

Primers and/or probes utilized in the disclosed methods need not have 100% homology to their targets to be effective, though in some embodiments, homology is substantially 100%. In some embodiments, primers and/or probes utilized herein have a homology to their respective target of about 50%, about 60%, about 70%, about 80%, about 85%, about 90%, about 95%, about 97%, about 98%, about 99%, about 99.9%, or up to substantially 100%. Some combinations of primers and/or probes may include primers and/or probes each with different homologies to their respective targets, and the homologies may be, for example, within a range with endpoints defined by any two of the foregoing values.

PCR and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as, e.g., loop-mediated isothermal amplification (“LAMP”), and other isothermal methods, such as those listed in Table 3, may require less or less extensive thermal cycling than does PCR, or require no thermal cycling. Such isothermal amplification methods are also contemplated for use with the assay compositions, kits, and methods described herein.

TABLE 3
Summary of optional isothermal amplification methods.

| | |
|---|---|
| NASBA | Nucleic acid sequence-based amplification (NASBA) is a |
| | method used to amplify RNA. |
| LAMP | Loop-mediated isothermal amplification (LAMP) is a single |
| | tube technique for the amplification of DNA. It typically |
| | uses 4-6 primers, which form loop structures to facilitate |
| | subsequent rounds of amplification. |
| HDA | Helicase-dependent amplification (HDA) uses the double- |
| | stranded DNA unwinding activity of a helicase to separate |
| | strands for in vitro DNA amplification at constant temperature. |
| RCA | Rolling circle amplification (RCA) starts from a circular DNA |
| | template and a short DNA or RNA primer to form a long single |
| | stranded molecule. |
| MDA | Multiple displacement amplification (MDA) is a technique |
| | that initiates when multiple random primers anneal to the |
| | DNA template and the polymerase amplifies DNA at constant |
| | temperature. |
| WGA | When MDA is used to amplify DNA from a whole genome of a |
| | cell it is called whole genome amplification (WGA). (Other |
| | methods of WGA include MALBAC, LIANTI, DOP-PCR.) |
| RPA | Recombinase polymerase amplification (RPA) is a low |
| | temperature DNA and RNA amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

Certain embodiments target RNA viruses such as SARS-CoV-2. SARS-CoV-2 has a single-stranded positive-sense RNA genome. In some embodiments, therefore, the amplification reaction (e.g., LAMP or PCR) can be combined with a reverse transcription (RT) reaction, such as in RT-LAMP or RT-PCR, to convert the RNA genome to a cDNA template. The cDNA template is then used to create amplicons of the target sequences in the subsequent amplification reactions. In some embodiments, the RT-PCR may be a one-step procedure using one or more primers and one or more probes as described herein. In some embodiments, the RT-PCR may be carried out in a single reaction tube, reaction vessel (e.g., “single-tube” or “1-tube” or “single-vessel” reaction). In some embodiments, the RT-PCR may be carried out in a multi-site reaction vessel, such as a multi-well plate or array. In some embodiments, RT and PCR are performed in the same reaction vessel or reaction site, such as in 1-step or 1-tube RT-qPCR. Suitable exemplary RTs can include, for instance, a Moloney Murine Leukemia Virus (M-MLV) Reverse transcriptase, SuperScript Reverse Transcriptases (Thermo Fisher Scientific), SuperScript IV Reverse Transcriptases (Thermo Fisher Scientific), or Maxima Reverse Transcriptases (Thermo Fisher Scientific), or modified forms of any such RTs.

In some embodiments, different assay products (e.g., amplicons from different variants) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety.

In some embodiments, the amplifying step can include performing qPCR, as that term is defined herein. qPCR is a sensitive and specific method for detecting and optionally quantifying amounts of starting nucleic acid template (e.g., coronaviral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. Other variations include an optical label and/or quencher positioned internally (i.e., not necessarily at the end) of the probe. Some probes may include more than one optical label and/or more than one quencher. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436, 134; 5,487,972; 5,658,751; 5,210,015; 5,487,972; 5,538,848; 5,618,711; 5,677, 152; 5,723,591; 5,773,258; 5,789,224; 5,801, 155; 5,804,375; 5,876,930; 5,994,056; 6,030,787; 6,084, 102; 6, 127, 155; 6, 171, 785; 6,214,979; 6,258,569; 6,814,934; 6,821,727; 7,141,377; and/or 7,445,900, all of which are hereby incorporated herein by reference in their entirety).

TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. The oligonucleotide probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

For example, a singleplex or multiplex qPCR can include a single TaqMan assay associated with a locus-specific sequence or multiple TaqMan assays respectively associated with a plurality of loci in a multiplex format. As a non-limiting example, a 4-plex reaction can include FAM (emission peak ˜517 nm), VIC (emission peak ˜551 nm), ABY (emission peak ˜580 nm), and JUN (emission peak ˜617 nm) dyes. In some embodiments, each dye is associated with one or more target sequences. In some embodiments, one or more dyes are quenched by a QSY quencher (e.g., QSY21). In some embodiments, each multiplex reaction allows up to 12 targets to be amplified and tracked real-time within a single reaction vessel. In some embodiments, up to 2, 4, 6, 8, 10, or 12 targets are amplified and tracked real-time within a single reaction vessel, using any combination of detectable labels disclosed herein or otherwise known to those of skill in the art. The aforementioned reporter dyes are optimized to work together with minimal spectral overlap for improved performance. Any combination of dyes described herein can additionally be combined with other dyes (e.g., Mustang Purple (emission peak ˜654 nm) or one or more Alexa Fluors (e.g., AF647 and AF676)), for use in monitoring fluorescence of a control or for use in a non-emission-spectrum-overlapping 5-plex assay. In addition, the QSY quencher is fully compatible with probes that have minor-groove binder quenchers.

Where multiple detection channels are utilized, it is desirable to minimize cross-talk between fluorescence reporters and select reporters that avoid excessive spectral overlap. One example of an assay that includes 5 detection channels incorporates the dyes FAM, ABY, VIC, and JUN, along with Mustang Purple (emission peak ˜654 nm) or an appropriate Alexa Fluor, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy-fluorescein (JOE)); 6-carboxy-1,4-dichloro-2′,7′-dichloro-fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red, Texas Red-X, among others as would be known to those of skill in the art. In certain embodiments, the use of a ROX dye improves precision of quantitation by reducing well to well differences from master mix volume (and/or other contributors to imprecision) in the PCR detection plate.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

The labeled amplicon (or labeled derivative thereof) can be detected using any suitable method such as, for example, electrophoresis, hybridization-based detection (e.g., microarray, molecular beacons, and the like), chromatography, NMR, and the like. In one exemplary embodiment, the labeled amplicon is detected using capillary electrophoresis. In another embodiment, the labeled amplicon is detected using qPCR.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), QuantStudio 7 RealTime PCR System (QS7), and/or QuantStudio 12K Flex System (QS12K), or a 7500 Real-Time PCR system, such as the 7500 Fast Dx system, from Thermo Fisher Scientific.

Some embodiments relate to kits containing primers and probes disclosed herein. Optionally, the kit can further include a master mix. In some embodiments, the master mix is TaqMan Fast Virus 1-Step Master Mix (Thermo Fisher Scientific, Catalog Nos. 44444432, 4444434, 4444436). In some embodiments, the master mix is TaqPath 1-Step RT-qPCR Master Mix, CG (Thermo Fisher Scientific, Catalog Nos. A15299, A15300). In other embodiments the master mix is TaqPath™ 1 Step Multiplex Master Mix (No ROX™) (Thermo Fisher Scientific, Catalog No. A48111, A28521, A28522, A28523). In some embodiments, the kit includes primers, probes and master mix sufficient to constitute a reaction mixture supporting amplification of one or more target regions from SARS-CoV-2.

### Examples

The following Examples may reference specific target nucleic acids, compositions, formulations, and/or process steps. It will be understood, however, that these Examples may be modified by using any of the alternative components described elsewhere herein.

### Example 1: Co-Linearity of SARS-CoV-2 and RNase P Amplification Efficiency

The TaqCheck™ SARS-CoV-2 Fast PCR Assay Kit was utilized to generate a standard curve plot for the N/S gene and for RNase P. Samples (2 replicates) were generated from spiked samples and comprised N/S gene IVT RNA from 1×10⁶ to 1×10¹ copies per reaction and RNase P IVT RNA from 1×10⁶ to 1×10¹ copies per reaction.

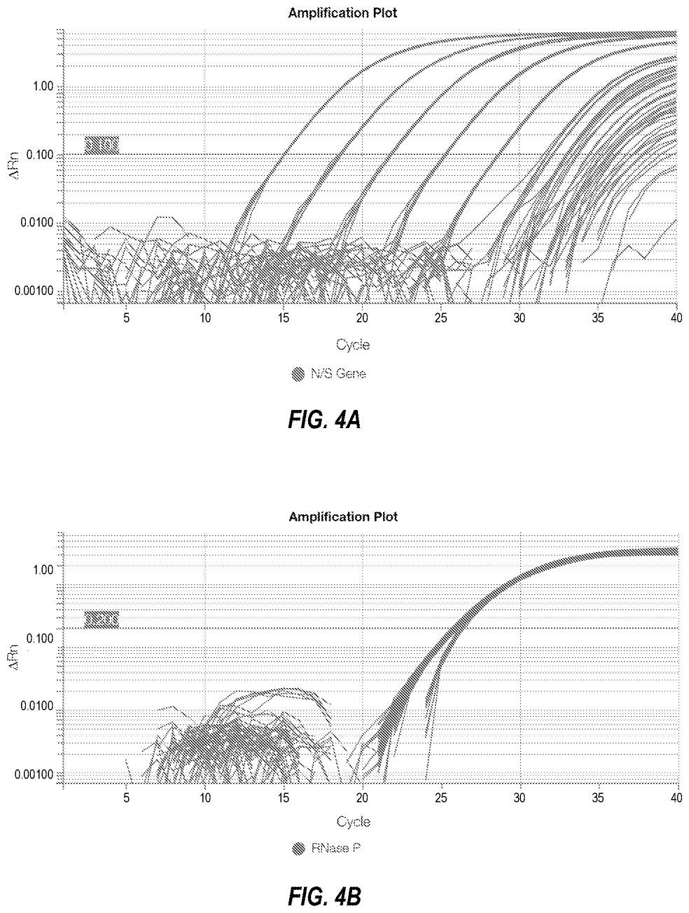

The results are shown in FIG. 3. As illustrated, the N/S gene and RNase P targets had similar PCR efficiency. Similarity in PCR efficiency beneficially allows the quantitation ratio of target nucleic acid (e.g., N/S gene) to endogenous nucleic acid (e.g., RNase P) to be effectively utilized for target titer correction. As shown, the efficiency of the N/S gene and the RNase P differs by only about 3-4%. Similarly, the slope (Cq/quantity) of the N/S gene and the RNase P differs by only about 3-4%.

### Example 2: Preparation of NP Sample Matrix

Nasopharyngeal (“NP”) samples were collected, pooled, and utilized as a sample matrix into which known concentrations of virion copies were spiked to generate samples. NP samples were purchased from multiple different vendors. Sample volumes ranged from 1-3 ml. When obtained from a vendor as a pre-pooled collection of samples, the total volume of any pool did not exceed 20 ml.

Each sample was tested (3 replicates) to confirm that samples were negative for the targets SARS-CoV-2, Flu A, Flu B, RSV A, and RSV B. Samples that tested negative for all targets were pooled to make a series of 200 ml pools. Following pooling, 20 replicates for each of the 200 ml pools were retested to confirm that samples were negative for all targets. TaqPath™ COVID-19, FluA/B RSV Combo Kit Instructions for Use (Thermo Fisher Scientific, MAN0019583) automated 400-μL sample input volume workflow was utilized to extract RNA. MS2 was added as positive control to each well and 5 negative controls were included per each RT-PCR plate. Using an Applied Biosystems™ 7500 Fast Real-Time PCR Instrument, the amplification conditions of Table 4 were applied (with 1.4° C./see ramp rate).

TABLE 4
Thermal Protocol

| Temperature | Time |
|---|---|
| 25° C. | 2 min - Hold |
| 53° C. | 10 min - Hold |
| 85° C. | 2 min - Hold |

| | | |
|---|---|---|
| 95° C. | 3 sec | 46 cycles |
| 60° C. | 30 sec | |

Results were analyzed to confirm negative results for all targets. Sample wells with clear amplification of MS2 positive control and no signal for any of the targets were designated negative. Sample wells that showed clear amplification of MS2 and clear or questionable amplification of one or more of the targets was considered a positive result. Only samples that were confirmed negative for all targets were used as sample matrix. Samples were also required to produce a MS2 Ct value of less than 28. Verified samples were then labeled and stored at −80° C.

### Example 3: qPCR Amplification of SARS-CoV-2 Serial Dilutions Using Example Assays

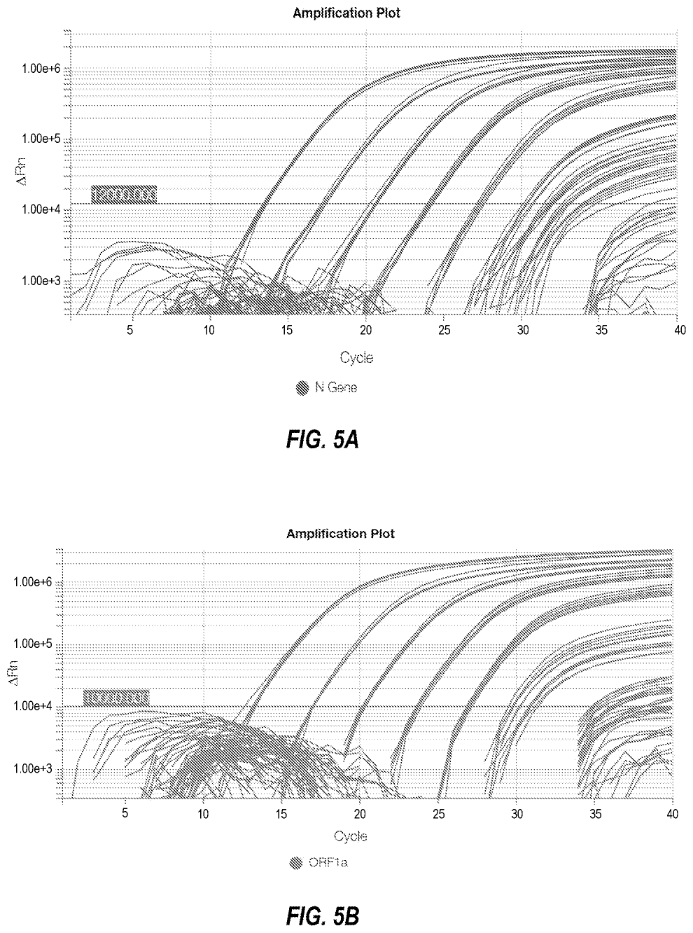

The NP sample matrix formed in Example 2 was utilized to form a serial dilution of SARS-CoV-2. The TaqCheck™ SARS-CoV-2 Fast PCR Assay Kit was utilized to perform amplification. Amplification was carried out using a protocol similar to that used in Example 2, but with the thermal protocol holding 85° C. for 10 minutes. Results for the N/S gene target and RNase P control are shown in FIGS. 4A and 4B, respectively. The results illustrate efficient amplification from 1.02×10⁸ to 1.33×10¹ copy/mL (6 replicates for each dilution). Detection was effective over at least 7 orders of magnitude of the dilution series. The RNase P was constant. The limit of detection (LoD) was approximately 1×10² copies/mL (100% detection rate at or above this level). The quantitation range was approximately 2×10² to 4×10⁷ copies/mL (the upper end being the highest level tested).

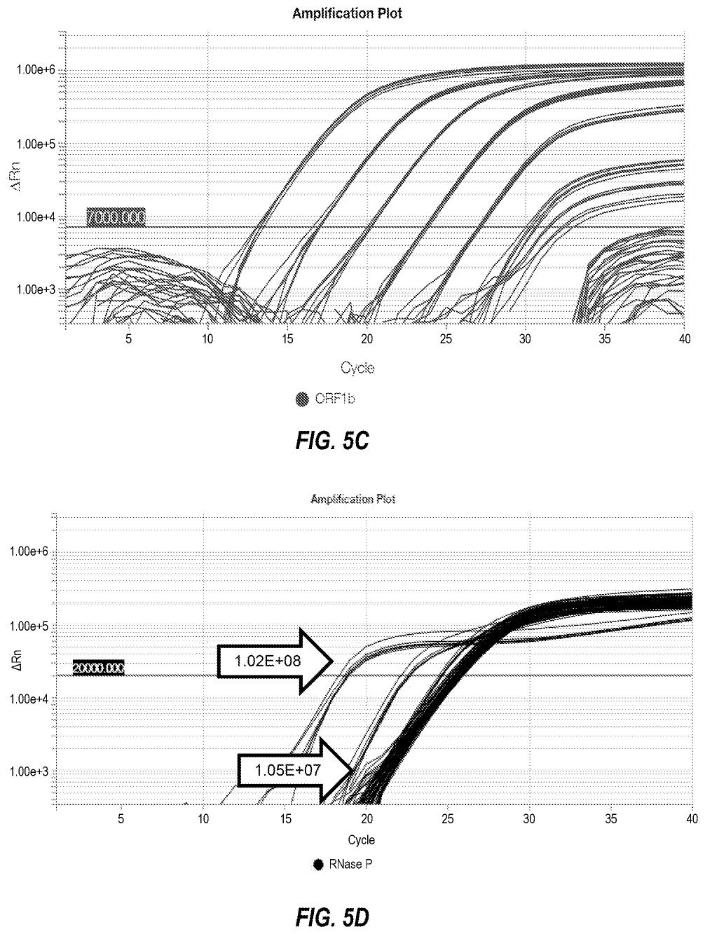

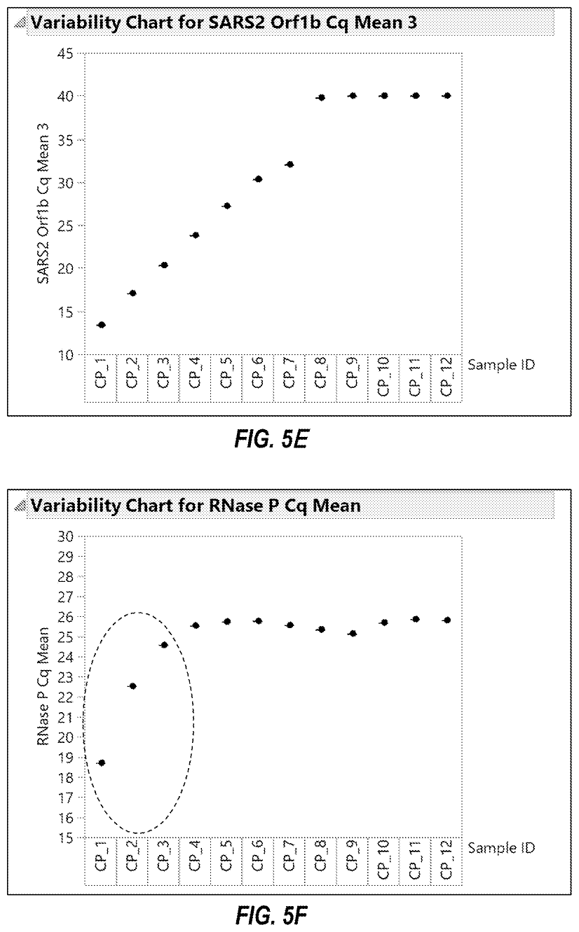

A similar test was performed using the TaqMan™ SARS-CoV-2 with RNase P Assay 2.0. Amplification results are illustrated in FIGS. 5A-5D. The results illustrate that there was cross-talk between the ABY dye channel (corresponding to Orf1b) and the JUN dye channel (corresponding to RNase P) at higher copy numbers. This is further illustrated by the variability charts of FIGS. 5E and 5F. Assays with such cross-talk are less preferred because the cross-talk effects the ratio intended to be utilized for normalizing measured target nucleic acid.

### Example 4: Normalization of Measured SARS-CoV-2 Quantities

Swab samples were taken from a subject over four time points (T0, T1, T2, and T3). The test samples were processed and subjected to amplification. Absolute quantity (AQ) of SARS-CoV-2 in each test sample was determined by comparing to a standard curve generated using a serial dilution of a control sample with known levels of SARS-CoV-2. RNase P levels were also determined in each test sample. An RQ value representing the ratio of SARS-CoV-2 to RNase P was determined for each test sample. The RQ of the T0 test sample was set to 1, and subsequent test samples were adjusted from the T0 baseline based on differences in their respective RQ values. Results are shown in Table 5.

TABLE 5
Target Quantification Corrected Based on RNase P Normalization

| | | | | | | Corrected | | |
|---|---|---|---|---|---|---|---|---|
| | Mean | Mean | | | COVID | COVID | Log | |
| | COVID | RNase P | Delta | | AQ | Quant. | (Quant.) | Log Diff. |
| | Cq | Cq | Cq | RQ | (copies/mL) | (copies/mL) | Diff. | from Ref |
| T₀ | 25.12 | 27.87 | −2.74 | 1.00 | 1.33E+05 | 1.33E+5 | 0.00 | 0.00 |
| T₁ | 25.68 | 28.07 | −2.38 | 0.78 | 8.82E+04 | 1.04E+5 | 0.07 | −0.11 |
| T₂ | 25.04 | 28.38 | −3.35 | 1.52 | 1.63E+05 | 2.02E+5 | 0.09 | 0.18 |
| T₃ | 29.96 | 29.15 | 0.82 | 0.09 | 4.63E+03 | 1.15E+4 | 0.40 | −1.06 |

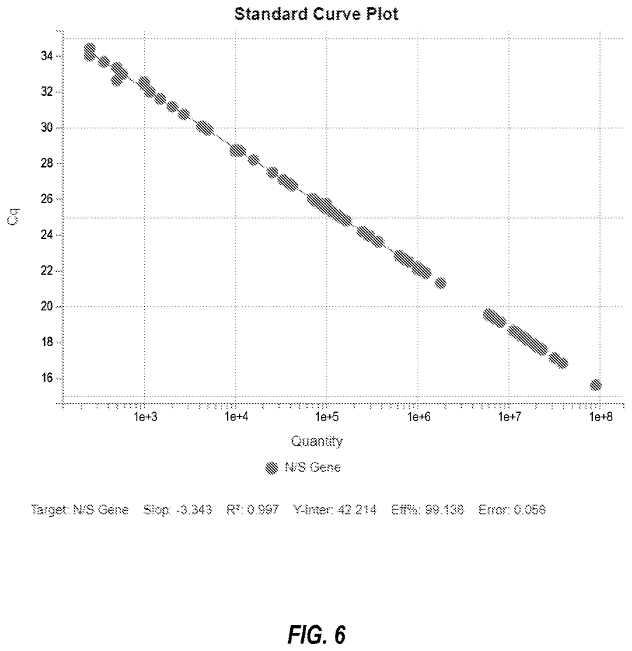

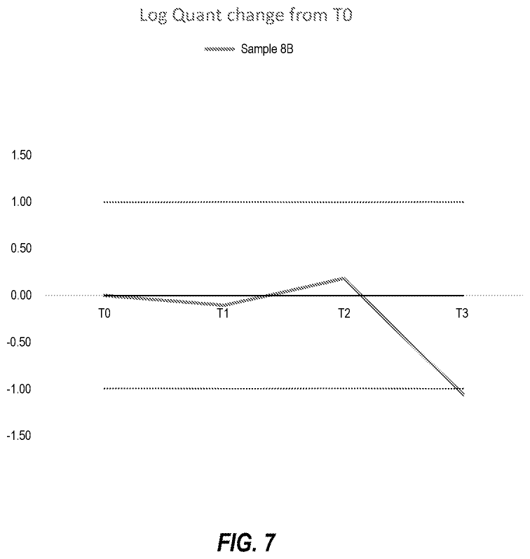

In Table 5, RQ represents a ratio of measured SARS-CoV-2 to measured RNase P. FIG. 6 illustrates the standard curve utilized to obtain “COVID AQ” values from the Cq values resulting from PCR. FIG. 7 illustrates the change in the Log (Quant.) over time from the initial T₀ time period. As shown, there are differences over time that the methods described herein are able to better quantify and thereby better provide monitoring of viral load changes over time (or changes in other target nucleic acids).

In this particular example, for a given time point (n), RQ(n) is calculated as:

$$R Q ( n ) = \frac{{( 1 + Eff . Target )}^{- ( C q ( n ) - C q ( 0 ) ) target}}{{( 1 + Eff . Control )}^{- ( C q ( n ) - C q ( 0 ) ) c ontrol}}$$

where “Eff. Target” is the PCR efficiency of the target nucleic acid, “Eff. Control” is the PCR efficiency of the control nucleic acid, (Cq(n)−Cq(0))_{target} is the difference between the measured Cq of the target nucleic acid at the nth sample and the Cq of the target nucleic acid at the initial “T₀” sample, and (Cq(n)−Cq(0))_{control} is the difference between the measured Cq of the endogenous control nucleic acid at the nth sample and the Cq of the control nucleic acid at the initial “T₀” sample.

What is claimed is:

1. A method for quantifying a target nucleic acid across multiple samples, the method comprising:
(a) providing two or more test samples each including the target nucleic acid;
(b) providing a set of control samples each having a known concentration of a control nucleic acid;
(c) amplifying at least a portion of the target nucleic acid in each of the test samples by subjecting each of the test samples to amplification conditions in the presence of target-specific primers;
(d) amplifying at least a portion of the control nucleic acid in each of the control samples by subjecting each of the control samples to amplification conditions in the presence of control primers;
(e) generating a standard curve using the results of amplifying the control nucleic acid;
(f) determining an absolute quantity (AQ) of the target nucleic acid in each of the test samples using the standard curve;
(g) amplifying an endogenous control nucleic acid in each of the test samples by subjecting each of the test samples to amplification conditions in the presence of endogenous sequence primers;
(h) determining a correction factor (RQ) for each of the test samples based on relative levels of endogenous nucleic acid in a respective one of the test samples; and
(i) determining a corrected quantity (corrected AQ) of the target nucleic acid in each of the test samples by normalizing the absolute quantity of the target nucleic acid using the correction factor of the respective one of the test samples.

2. The method of claim 1, wherein the two or more test samples are each derived from the same subject.

3. The method of claim 2, wherein the two or more test samples are obtained at different times and/or from different locations of the subject.

4. The method of claim 3, wherein at least two of the different times are separated by a time period of 1 hour, 2 hours, 3 hours, 6 hours, 12 hours, 24 hours, 48 hours, 72 hours, 96 hours, one week, two weeks, three weeks, four weeks, six weeks, eight weeks, ten weeks, three months, four months, five months, six months, or a time period range with endpoints defined by any two of the foregoing values.

5. The method of any one of claims 1-4, wherein the target nucleic acid is a viral nucleic acid.

6. The method of claim 5, wherein the target nucleic acid is a SARS-CoV-2 nucleic acid.

7. The method of any one of claims 1-6, wherein the test samples are derived from swab samples.

8. The method of claim 7, wherein the swab samples are nasal swab samples.

9. The method of claim 8, wherein each of the nasal swab samples is from a same nostril of the subject.

10. The method of any one of claims 7-9, further comprising extracting the target nucleic acid from the swab samples prior to subjecting each of the test samples to amplification conditions.

11. The method of any one of claims 1-10, wherein the control nucleic acid in each of the control samples comprises a nucleic acid comprising the target nucleic acid.

12. The method of claim 11, wherein the control nucleic acid in each of the control samples comprises a whole or partial viral genome.

13. The method of any one of claims 1-12, wherein the control nucleic acid in each of the control samples is capable of being amplified with the target-specific primers.

14. The method of any one of claims 1-13, wherein the control primers and the target-specific primers are the same.

15. The method of any one of claims 1-14, wherein the endogenous sequence primers are specific for RNase P.

16. The method of any one of claims 1-15, wherein the target-specific primers are specific to one or more of the Orf1a gene, the Orf1b gene, the N gene, or the S gene of SARS-CoV-2.

17. The method of any one of claims 1-16, wherein the test samples exclude blood samples.

18. The method of any one of claims 1-17, wherein the test samples are nasopharyngeal or oropharyngeal samples.

19. The method of any one of claims 1-18, further comprising establishing a baseline correction factor for a first test sample associated with a first time point, and determining subsequent correction factors for subsequent test samples relative to the baseline correction factor.

20. The method of claim 19, further comprising determining a corrected quantity of the subsequent test samples relative to the first test sample to illustrate relative change in target nucleic acid load of the test samples over time.

21. The method of any one of claims 1-20, wherein the amplifying of the at least a portion of the target nucleic acid in each of the test samples comprises a reverse transcription reaction.

22. The method of any one of claims 1-21, wherein the amplifying of the at least a portion of the control nucleic acid in each of the control samples comprises a reverse transcription reaction.

23. The method of any one of claims 1-22, wherein the amplifying of the endogenous nucleic acid in each of the test samples excludes a reverse transcription reaction.

24. The method of any one of claims 1-23, wherein amplification of the control nucleic acid in each of the control samples and amplification of the endogenous control nucleic acid in each of the test samples have substantially similar efficiency.

25. The method of claim 24, wherein amplification of the control nucleic acid in each of the control samples and amplification of the endogenous control nucleic acid in each of the test samples have efficiencies that differ by no more than about 6%, no more than about 5%, or no more than about 4%.

26. The method of claim 24 or claim 25, wherein an efficiency plot of amplification of the control nucleic acid in each of the control samples and an efficiency plot of amplification of the endogenous control nucleic acid in each of the test samples have slopes (Cq/quantity) that differ by no more than about 6%, no more than about 5%, or no more than about 4%.

27. A method for quantifying a target viral nucleic acid from a sample, the method comprising:
(a) providing two or more test samples each including the target viral nucleic acid;
(b) providing a set of control samples each having a known concentration of the target viral nucleic acid;
(c) amplifying at least a portion of the target viral nucleic acid in each of the test samples by subjecting each of the test samples to amplification conditions in the presence of target-specific primers;
(d) amplifying at least a portion of the target viral nucleic acid in each of the control samples by subjecting each of the control samples to amplification conditions in the presence of the target-specific primers;
(e) generating a standard curve using the results of amplifying the target viral nucleic acid in each of the control samples;
(f) determining an absolute quantity of the target viral nucleic acid in each of the test samples using the standard curve;
(g) amplifying an endogenous nucleic acid in each of the test samples by subjecting each of the test samples to amplification conditions in the presence of endogenous sequence primers;
(h) determining a correction factor for each of the test samples based on relative levels of endogenous nucleic acid in a respective one of the test samples; and
(i) determining a corrected quantity of the target viral nucleic acid in each of the test samples by normalizing the absolute quantity of the target viral nucleic acid using the correction factor for the respective one of the test samples.

28. The method of claim 27, wherein the two or more test samples are each derived from the same subject.

29. The method of claim 28, wherein the two or more test samples are obtained at different times.

30. The method of claim 29, wherein at least two of the different times are separated by a time period of 1 hour, 2 hours, 3 hours, 6 hours, 12 hours, 24 hours, 48 hours, 72 hours, 96 hours, one week, two weeks, three weeks, four weeks, six weeks, eight weeks, ten weeks, three months, four months, five months, six months, or a time period range with endpoints defined by any two of the foregoing values.

31. The method of any one of claims 27-30, wherein the target viral nucleic acid is a SARS-CoV-2 nucleic acid.

32. The method of any one of claims 27-31, wherein the test samples are derived from swab samples.

33. The method of claim 32, wherein the swab samples are nasal swab samples.

34. The method of claim 33, wherein each of the nasal swab samples is from a same nostril of the subject.

35. The method of any one of claims 32-34, further comprising: extracting the target nucleic acid from the swab samples prior to subjecting each of the test samples to the amplification conditions.

36. The method of any one of claims 27-35, wherein the endogenous sequence primers are specific for RNase P.

37. The method of any one of claims 27-36, wherein the target-specific primers are specific to one or more of the Orf1a gene, the Orf1b gene, the N gene, or the S gene of SARS-CoV-2.

38. The method of any one of claims 27-37, wherein the test samples exclude blood samples.

39. The method of any one of claims 27-38, wherein the test samples are nasopharyngeal or oropharyngeal samples.

40. The method of any one of claims 27-39, further comprising: establishing a baseline correction factor for a first test sample associated with a first time point; and determining subsequent correction factors for subsequent and/or additional test samples relative to the baseline correction factor.

41. The method of claim 40, further comprising: determining a corrected quantity of the subsequent test samples relative to the first test sample to illustrate relative change in target nucleic acid load of the test samples over time.

42. The method of any one of claims 27-41, wherein the amplifying of at least a portion of the target nucleic acid in each of the test samples comprises a reverse transcription reaction.

43. The method of any one of claims 27-42, wherein the amplifying of at least a portion of the target nucleic acid in each of the control samples comprises a reverse transcription reaction.

44. The method of any one of claims 27-43, wherein the amplifying of the endogenous nucleic acid in each of the test samples excludes performing a reverse transcription reaction.

45. The method of any one of claims 27-44, wherein the amplifying of the target nucleic acid in each of the control samples and the amplifying of the endogenous nucleic acid in each of the test samples have substantially similar efficiency.

46. The method of claim 45, wherein an efficiency plot of amplification of the target nucleic acids in each of the control samples and an efficiency plot of amplification of the endogenous nucleic acid in each of the test samples have slopes (Cq/quantity) that differ by no more than about 6%, no more than about 5%, or no more than about 4%.
